(19) United States
(12) Patent Application Publication
(10) Pub. No.: US 2024/0153634 A1
(43) Pub. Date: May 9, 2024

(54) METHOD AND SYSTEM FOR PERFORMING DATA STRUCTURING AND GENERATING HEALTHCARE INSIGHTS

(71) Applicant: Srinka GHOSH, San Francisco, CA (US)

(72) Inventor: Srinka GHOSH, San Francisco, CA (US)

(21) Appl. No.: 18/386,365

(22) Filed: Nov. 2, 2023

Related U.S. Application Data
(60) Provisional application No. 63/421,947, filed on Nov. 2, 2022.

(51) Int. Cl. G16H 50/20; G06F 40/40; G16H 15/00

(52) U.S. Cl. CPC G16H 50/20; G06F 40/40; G16H 15/00

(57) ABSTRACT

A method for performing data structuring on unstructured medical data and generating healthcare insights. The method may include receiving the unstructured medical data pertaining to at least one patient and at least one physician; transforming the received unstructured medical data into structured data using a data-centric artificial intelligence (DCAI); labeling the structured data and performing hidden structure discovery to establish data equivalence of the labeled data; and providing the hidden structure discovered data as input to a model-centric artificial intelligence (MCAI) engine to generate the healthcare insights.

### CROSS-REFERENCE TO RELATED APPLICATIONS

This application claims priority under 35 USC § 119(a) to U.S. Provisional Application No. 63/421,947, filed on Nov. 2, 2022, the contents of which are incorporated herein by reference in their entireties.

### BACKGROUND

### Field

The present disclosure is generally directed to a method and a system for performing data structuring on unstructured healthcare data, creating adaptive clinical decisions, and generating healthcare insights.

### Related Art

Healthcare data application and storage have been on the rise since the early 2010s. By 2018, the healthcare enterprise data sphere amounted to roughly 1.2 ExaBytes. By 2025, this number is expected to grow by another third. The growth is largely driven by an increase in digital device interactions. In 2020, the number was 1400 per person (PP)/day, and by 2025, the number is expected to rise to 4000 PP/day.

With the COVID-19 outbreak back in 2019, the digisphere was turbocharged by the watershed moment. In 2019, most physicians spent on average 1-5 hours a day, outside of their regular work hours, documenting clinical care in paper and electronic medical record (EMR) systems. In 2017, there was generation of 80 megabytes of EMR and imaging data per year per patient. In 2018, healthcare was contributing 1.2 ExaBytes of data globally and was projected to have a compound annual growth rate (CAGR) of 36% between 2018-2025.

Medication shortages/differential access to medication can have adverse effects on patient outcomes. Many shortages are in the therapeutic areas of oncology, transplant, pediatrics, etc. Shortages can adversely impact economic, clinical, and humanistic outcomes, ranging from delayed care, hospital readmissions, to longer admission periods to patient mortality. Which can distinctly impact care-giver burnouts.

### SUMMARY

Aspects of the present disclosure involve an innovative method for performing data structuring on unstructured healthcare data and generating healthcare insights. The method may include receiving first structured healthcare data and the unstructured healthcare data pertaining to at least one patient and at least one physician; transforming the received unstructured healthcare data into second structured healthcare data using a data-centric artificial intelligence (DCAI); labeling the first structured healthcare data and the second structured healthcare data, and performing hidden structure discovery to establish data equivalence of the labeled data; and providing the hidden structure discovered data as input to a model-centric artificial intelligence (MCAI) engine to generate the healthcare insights.

Aspects of the present disclosure involve an innovative non-transitory computer readable medium, storing instructions for performing data structuring on unstructured healthcare data and generating healthcare insights. The instructions may include receiving first structured healthcare data and the unstructured healthcare data pertaining to at least one patient and at least one physician; transforming the received unstructured healthcare data into second structured healthcare data using a data-centric artificial intelligence (DCAI); labeling the first structured healthcare data and the second structured healthcare data, and performing hidden structure discovery to establish data equivalence of the labeled data; and providing the hidden structure discovered data as input to a model-centric artificial intelligence (MCAI) engine to generate the healthcare insights.

Aspects of the present disclosure involve an innovative server system for performing data structuring on unstructured healthcare data and generating healthcare insights. The server system may include receiving first structured healthcare data and the unstructured healthcare data pertaining to at least one patient and at least one physician; transforming the received unstructured healthcare data into second structured healthcare data using a data-centric artificial intelligence (DCAI); labeling the first structured healthcare data and the second structured healthcare data, and performing hidden structure discovery to establish data equivalence of the labeled data; and providing the hidden structure discovered data as input to a model-centric artificial intelligence (MCAI) engine to generate the healthcare insights.

Aspects of the present disclosure involve an innovative system for performing data structuring on unstructured healthcare data and generating healthcare insights. The system can include means for receiving first structured healthcare data and the unstructured healthcare data pertaining to at least one patient and at least one physician; means for transforming the received unstructured healthcare data into second structured healthcare data using a data-centric artificial intelligence (DCAI); means for labeling the first structured healthcare data and the second structured healthcare data, and performing hidden structure discovery to establish data equivalence of the labeled data; and means for providing the hidden structure discovered data as input to a model-centric artificial intelligence (MCAI) engine to generate the healthcare insights.

### BRIEF DESCRIPTION OF DRAWINGS

A general architecture that implements the various features of the disclosure will now be described with reference to the drawings. The drawings and the associated descriptions are provided to illustrate example implementations of the disclosure and not to limit the scope of the disclosure. Throughout the drawings, reference numbers are reused to indicate correspondence between referenced elements.

FIG. 1 illustrates an example data interaction 100 for an ecosystem A×P, in accordance with an example implementation.

FIG. 2 illustrates an example evolution 200 of an ecosystem A×P, where A is pAtient and P is Physician, with respect to a patient's life, in accordance with an example implementation.

FIG. 3 illustrates example supersets 300 of ecosystems, in accordance with an example implementation.

FIG. 4 illustrates elements 400 of an example interaction I graph for an A×P ecosystem, in accordance with an example implementation.

FIG. 5 illustrates compact representation table 500 of nodes in an interaction I graph, in accordance with an example implementation.

FIGS. 6(A)-(B) illustrate an example interaction I stratification table 600 for the A×P ecosystem, in accordance with an example implementation.

FIG. 7 illustrates an example data structuring process 700 using unstructured data, in accordance with an example implementation.

FIG. 8 illustrates example paradigms 800 associated with the modeling of diagnosis, in accordance with an example implementation.

FIG. 9 illustrates an example ecosystem 900 of healthcare data, in accordance with an example implementation.

FIG. 10 illustrates example data inputs 1000 for a model-centric AI, in accordance with an example implementation.

FIG. 11 illustrates tagging process 1100 of patients and physicians using Brick B, in accordance with an example implementation.

FIG. 12 illustrates an example data lake 1200 with patient-centric micro-lakes and physician-centric micro-lakes, in accordance with an example implementation.

FIG. 13 illustrates an example patient-centric micro-lake generation approach 1300, in accordance with an example implementation.

FIG. 14 illustrates an example physician-centric micro-lake generation approach 1400, in accordance with an example implementation.

FIG. 15 illustrates example models 1500 of multi-physician diagnosis thesaurus, in accordance with an example implementation.

FIGS. 16(A)-(C) illustrate example insights generated by Brick R, in accordance with an example implementation.

FIG. 17 illustrates an example computing environment with an example computer device suitable for use in some example implementations.

FIGS. 18(A)-(B) illustrate a flywheel representing the healthcare data conversion and insight generation system.

FIG. 19 illustrates an example process flow 1900 for generating healthcare insights and medical recommendation, in accordance with an example implementation.

### DETAILED DESCRIPTION

The following detailed description provides details of the figures and example implementations of the present application. Reference numerals and descriptions of redundant elements between figures are omitted for clarity. Terms used throughout the description are provided as examples and are not intended to be limiting. For example, the use of the term “automatic” may involve fully automatic or semi-automatic implementations involving user or administrator control over certain aspects of the implementation, depending on the desired implementation of one of the ordinary skills in the art practicing implementations of the present application. Selection can be conducted by a user through a user interface or other input means, or can be implemented through a desired algorithm. Example implementations as described herein can be utilized either singularly or in combination and the functionality of the example implementations can be implemented through any means according to the desired implementations.

Example implementations provide for a platform that equalizes access to high-value healthcare notwithstanding barriers of location, income, and education among other socioeconomic conditions. Example implementations profile a patient's health journey as a function of time (a continuum), with a health snapshot available at any point in time (a discrete variable) to any physician, e.g., an automated patient tracker or automated chart reviewer. Patterns of physician's treatment paradigm can be identified via triangulation of patient symptoms, tests, diagnosis, medication, etc. This may involve optimizing and streamlining matching of patients with primary care physicians (PCP), specialists, emergency room (ER)/emergency department (ED) physicians, etc.

Example implementations also utilize an automated treatment recommendation engine personalized to a given physician. For example, charting out an optimized care-pathway for a patient in consideration of physician treatment preferences. In addition, an Rx recommendation engine can also be provided to generate alternative Rx paradigms. The recommendation engines can be used to generate differential diagnosis in a comprehensive manner for ER/ED for more accurate, expedient and efficient patient care, which in turn reduces misses, delayed diagnosis, and/or mis-diagnosis in the ER. Similarly, the recommendation engines can be utilized to generate differential diagnosis in non-emergent healthcare or PCP settings.

Example implementations provide a collaborative framework where physicians/caregivers can provide input on a patient. The information provided can be synthesized using the collaborative framework and actionable insights can be recommended back to the care providers. For example, this can be applied to an ED where care is coordinated across multiple medical stakeholders including physicians, nurse practitioners, schedulers, imaging specialists, pharmacists, etc.

Example implementations provide for the creation of a global forum where symptoms-to-diagnoses-to-actionable-insights can be socialized. For example, obstetricians and gynecologists (OB-GYNs) in parts of the world with limited resources can access the forum for consulting purposes for ideas exchange. This is also extensible to organizations such as Doctors without Borders enabling connectivity with physicians who have access to limited technical resources to their counterparts who might have had past experiences with similar resource scarcity as well as experts who are not resource deprived.

In addition to providing data redundancy in EMR, example implementations also provide optimized clinical trial outcomes through use of a pipeline for patient selection in clinical trials. The platform can provide prognostic and predictive enrichment to identify patients for clinical trial matching.

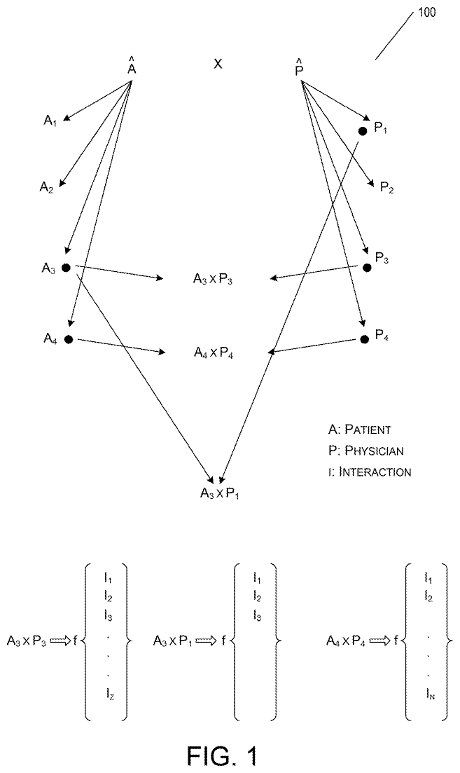

FIG. 1 illustrates an example data model 100 for an ecosystem A×P, in accordance with an example implementation. A×P is a unique ecosystem shared between a patient and a physician. “A” represents the patient identifier and “P” represents the physician identifier. As illustrated in FIG. 1, using patient A₃ as an example, the ecosystem A₃×P₃ captures interactions between patient A₃ and physician P₃. A clinical decision support system (CDSS) is created to capture the interactions between patients and physicians and generate the various ecosystems capturing the tracked interactions. If a patient interacts with multiple physicians, then there will be multiple environments for each unique A×P. Taking patient A₃ as example, A₃ interacts with two different physicians P₁ and P₃ and this results in two unique ecosystems as defined by A₃×P₁ and A₃×P₃.

The CDSS captures interactions between the two distinct players of a patient and a physician. Each A×P ecosystem is defined by interactions I. The ensuing details provide an example of the underlying data model supporting the CDSS. Specifically, each A×P comprises of a superposition of signal from multiple interactions. For example, A₃×P₁ is a superposition of I₁, I₂ and I₃. The core elements of an interaction I is defined by patient A, physician P, and formalized by a time-stamp (ρ) which uniquely indexes the point in time along the patient's health journey to the interaction with the physician. I is represented by:

I→f[A,P,time-stamp]

Any given A×P supports multiple interactions I between specific A and P and is modelled as a superposition of signal. In some example implementations, status of the patient and physician can evolve over time in the sequence of interactions, as represented by:

Sequence of interactions: Iₚ→f[Aₚ,Pₚ,(ρ)]

(A×P)_{ρ}=Σ_{ρ=1}^{z}Iₚ

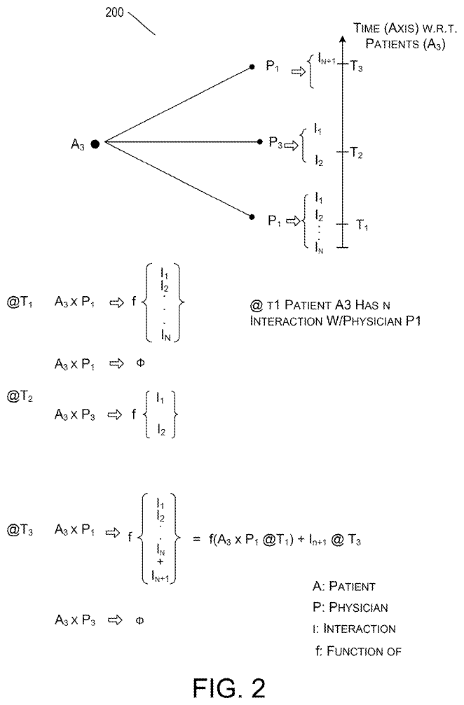

Any given ecosystem evolves over time. The definition of the ecosystem can be discontinuous with regards to the patient's life(time). FIG. 2 illustrates an example evolution 200 of an ecosystem A×P with respect to a patient's life, in accordance with an example implementation. The y-axis represents time change with respect to the patient's life(time). As illustrated in FIG. 2, at time T₁, A₃ visits P₁ over multiple interactions (I₁ . . . Iₙ), with T₁ being a time range earlier in time in comparison to T₂ and T₃. This results in the definition of A₃×P₁ at T₁. At T₂, A₃ visits P₃ over 2 interactions (I₁, I₂), defined as A₃×P₃ at T₂. Since no interaction exists at T₂ with P₁, there's no further evolution of A₃×P₁ with respect to T₂. At T₃, patient A₃ visits P₁ at T₃ over interaction Iₙ₊₁, a time later in comparison to both T₁ and T₂. A₃×P₁ now evolves from its prior state at T₁ to include the Iₙ₊₁ interaction and there is further evolution of A₃×P₁ due to interactions at T₃. No further evolution of A₃×P₃.

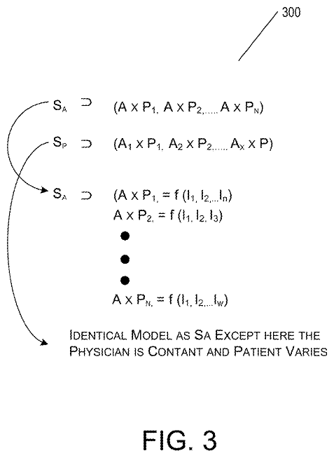

FIG. 3 illustrates example supersets 300 of ecosystems, in accordance with an example implementation. The platform/CDSS supports the structure of supersets of A×P ecosystems, which enables both a patient centric model (S_{A}) and a physician centric model (S_{P}).

Superset S_{A}: The superset where the patient remains constant. The multiplicity is due to introduction of different physicians (P) the patient might interact with over their lifetime. Superset S_{A} is represented by:

S_{A}⊃(A×P₁,A×P₂ . . . ,A×Pₙ), where A: constant and P: variable;

The equation is a superposition of signal from multiple interactions across multiple ecosystems involving the same patient: S_{A}⊃(A×P₁=f [symbol] I₃ [symbol] , A×P₂=f [symbol] P₂, . . . [symbol] . . . , A×Pₙ=f [symbol] Pₙ, . . . [symbol] ).

Superset S_{P}: The superset where the physician remains constant. The multiplicity is due to introduction of different patients (A) the physician treats. Superset S_{P} is represented by:

Sₚ⊃(A₁×P,A₂×P . . . ,Aₙ×P), where A: variable and P: constant;

The equation is a superposition of signal from multiple interactions across multiple ecosystems involving the same physician but different patients: S_{P}⊃(A₁×P=f [symbol] I₃ [symbol] , A₂×P=f [symbol] A₂, . . . [symbol] . . . , Aₙ×P=f [symbol] Aₙ, . . . [symbol] ).

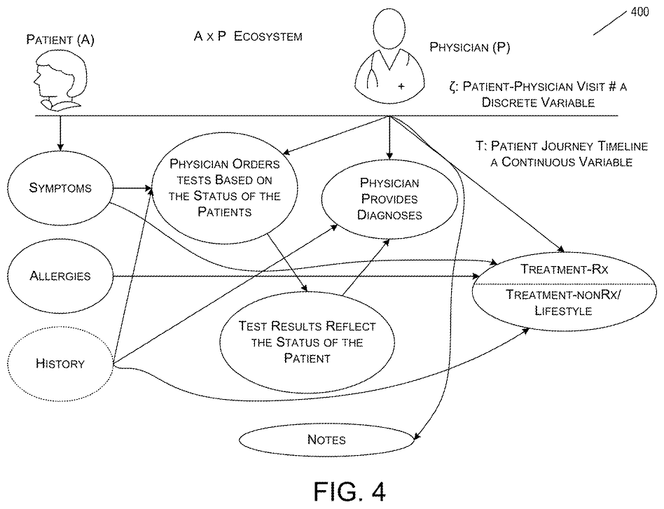

FIG. 4 illustrates elements 400 of an example interaction I graph for an A×P ecosystem, in accordance with an example implementation. As illustrated in FIG. 4, a number of nodes exist in the A×P interaction I topography. Each node is owned by one of the players (e.g., patient, physician, etc.), or both players, if present at the owned node, exist as participant. While a node must have an owner, the same node is not required to have any participant. In some example implementations, a node is not required to be owned by any player. If a node is ownerless, then the content of the node will not contribute to the interaction topology. However, the ownership of a previously unowned node can be changed to an appropriate owner if the information content of the node adds value to the model.

A node is a primary node if it does not inherit or stem from a prior node. A node is a derived node if it inherits or stems from a prior node. In addition, a node can inherit or stem from one or more prior nodes. If a node inherits from multiple prior nodes, one of the multiple prior nodes can have stronger influence that the others, and this node is referred to as a driver node. Node linkage complies with chronology of prior nodes and elements or attributes of prior node(s) must be inherited in totality.

A function f annotated to a node explicitly states the inputs in its parameter definition. In case of inheritance, decomposition of each component function will enable reduction to a final function F representing the atomic elements of the interaction I, such as I_{ρ}→f[A_{ρ},P_{ρ}, (ρ)].

Annotation reduced to the atomic elements of I will identify node-linkages and ownership. Ownership identifies who owns or initiated the node, as well as weighted impact on the content of the node (e.g. weight/impact by each of the players on the response from the node, etc.).

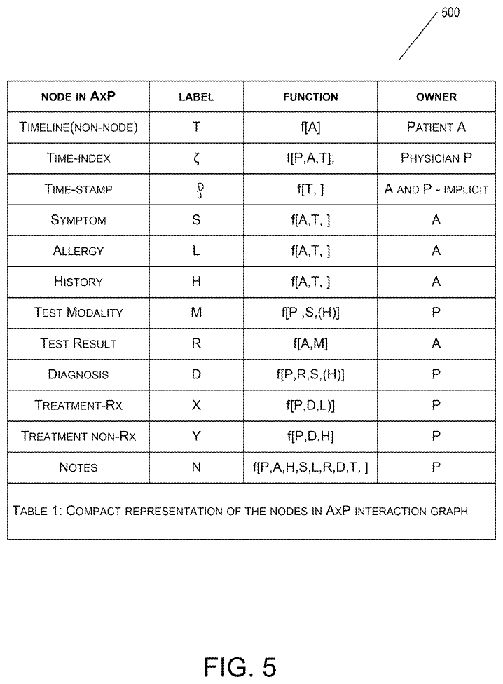

FIG. 5 illustrates compact representation table 500 of nodes in an interaction I graph, in accordance with an example implementation. The A×P interaction incorporates two time concepts to create a time-stamp (ρ):
1. Timeline T: a continuum along the reference frame of the patient or patient's lifetime, represented by x-axis as illustrated in FIG. 4; and
2. Time-index τ: an index of the time-encounter specific to A and P along timeline T of patient. Time-stamp is represented by (ρ)→f[T,τ]. For example: τₗ represents first encounter in Aᵢ×Pⱼ in the interaction between Aᵢ and Pⱼ.
   The index resets for every new physician or patient, for example: Pₗ: τₗ represents first encounter in Aᵢ×Pₗ in the interaction between Aᵢ and Pₗ; and Aᵢ: τₗ represents encounter in Aᵢ×Pₗ in the interaction between Aᵢ and Pₗ.

As illustrated in FIG. 5, Patient A exclusively owns T→f[A], a non-node in the ecosystem. The underline indicates ownership by the underlined player. Physician P owns the time index, a primary node which is a sequential index tracking the patient-physician encounter, where the patient is a participant. The time index is represented by τ→f[P,A,T]. Patient A owns the following primary nodes with no participant:
1. Symptoms: S→f[A,T,τ]→F_{S}[A¹,P⁰,T,τ] patient A has symptom(s) S at time T during encounter τ with physician P. Representationally, S is a 1D array with s≥1 element(s), since patient A can have multiple symptoms at time-stamp ρ;
2. Allergies: L→f[A,T,τ]→F_{A}[A¹,P⁰,T,τ] A has allergies L at T, τ. Representationally, L is a 1D array with 1≥1 element(s); and
3. History: H→f[A,T,τ]→F_{H}[A¹,P⁰,T,τ] A has history H at T, τ. Representationally, H is a composite of patient narratives, EMR/legacy paper records, data from prior physician(s), etc. It is expected to have a varying degree of verifiability and accuracy. It is critical to establish the provenance of this information since that will dictate complete/partial inclusion of this data. In some example implementations, H may also include impacts (e.g., mood change, depression, etc.) of stressors (e.g., isolation, austere environments, space radiation, etc.)
   Only patient A has any ownership of the identified primary nodes, since physician has no ownership of or impact on the content of the node (P⁰). Node-linkages of labels S/L/H to patient A are illustrated in FIG. 5.

As illustrated in FIG. 5, physician P owns the derived node test modality (M): M→f[P, S(H)]. For example, physician P orders a set of tests M based on patient symptoms S while taking patient history H into consideration at time-stamp ρ. Elements of (φ→f[T,τ] are not explicitly stated, since it's inherited via S. The viability of history H is variable as discussed above. Placement of H in parenthesis signifies its inclusion/contribution might be partial. Representationally, M is a one-dimension array with m≥1 element(s) since physician P can request a palette of tests M. Since physician P is responsible for ordering/assigning the tests, physician P is the owner of node M. Node M inherits node S(H) and the inheritance signifies that M is a function of patient A (via S)—since his/her symptoms S—at time T, during the encounter τ with physician P dictates the test choices. The annotation of this derived node can be reduced to the following atomic elements:

M→f[P,S,(H)]→f[P,S,(H)]→f[P,f[A,T,τ]]→F_{M}[P^{l},A^{l},T,τ]:designates a decomposition function where {S,(H)}→f[A,T,τ]

Since physician P owns the node and patient A owns node S, both patient A and physician P have equal impact on the node. This is exhibited through the node-linkages of M←S(H)←A.

As illustrated in FIG. 5, patient A owns the derived node test result (R): R→f[A,M]. Test result R reflect the status of patient A with symptom S. Representationally, R is a one-dimensional array with r≥1 element(s) since physician P can request m≥1 test with a one-to-one mapping between tests & results. The annotation of this derived node can be reduced to the following atomic elements:

R→f[A,M]→f[A{M}]→f[A,f[P,S,(H)]]→[A,f[P,{S,(H)}]]→f[A,f[P,f[A,T,τ]]]→F_{R}[A²,P^{l},T,τ]

Since patient A owns the node S(H) and both patient A and physician P have equal impact on the content of node M, overall, patient A has higher impact on the test result node. This is exhibited through the node-linkages of R←M←S(H)←A.

As illustrated in FIG. 5, physician P owns the derived node diagnosis (D): D→f[P,R,S(H)], which inherits from three prior nodes. Physician P diagnoses a patient based on the result R from test M ordered in response to symptom S taking into consideration of patient history H during encounter τ at time T. While the linkage R←M←S(H) inherits S(H) via M, there might be S(H) that the physician does not test for, therefore a direct connectivity with symptom (S) and history (H) is also established. Representationally, D is a one-dimensional array with d=1 element or a nested element. The annotation of derived nodes can be reduced to the atomic elements:

D→f[P,R,S(H)]→f[P,{R},{S(H)}]→f[P,f[A,M],f[A,T,τ]]→f[P,f[A,{M}],f[A,T,τ]]→f[P,f[A,f[P,S,(H)]],f[A,T,τ]]→f[P,f[A,f[P,δ{S,(H)}]],f[A,T,τ]]→f[P,f[A,f[P,f[A,T,ι]]→f[A,T,ι]]→F_{D}[P²,A³,T,τ]

While physician P owns the node, patient A has an overall higher impact on node via both direct (non-tested symptom S) and indirect (tested symptom S) connectivity to the content of the node. This is exhibited through the node-linkages of D←R←S(H)←A and D←S(H)←A, compactly represented as D←R←S(H)←A.

As illustrated in FIG. 5, physician P owns the derived node treatment-Rx (X): X→f[P,D,L], which inherits from three prior nodes. Physician P suggests Rx treatment(s) X for patient A based on the diagnosis D while taking allergies L into consideration. X is in response to S(H) during the encounter τ at time T. Representationally, X is a two-dimensional array with x≥1 element(s) since physician P can prescribe multiple Rx with different dosage(s). The annotation of derived nodes can be reduced to the atomic elements:

X→f[P,D,L]→f[P,{D},{L}]→f[P,f[P,R,S,(H)],f[A,Tτ]]→f[P,f[P,{R},{S,(H)}],f[A,T,τ]]→f[P,f[P,f[A,M],f[A,T,τ]],f[A,T,τ]]→f[P,f[P,f[A,{M}],f[A,T,τ]],f[A,T,τ]]→f[P,f[P,f[A,f[P,S,(H)]],f[A,T,τ]],f[A,T,τ]]→f[P,f[P,f[A,f[P,{S,(H)}]],f[A,T,τ]],f[A,T,τ]]→f[P,f[P,f[A,f[P,f[A,T,τ]]],fA,T,τ]],f[A,T,τ]]→Fx[P³,A⁴,T,τ]

Patient has an overall higher impact on node than physician P via diagnosis D and the associated node inheritances that are owned by patient A and allergy L. This is exhibited through the node-linkages of X←D←R←S(H)←A also X←L←A.

As illustrated in FIG. 5, physician P owns the derived node treatment-non-Rx (Y): Y→f[P, D, H], which inherits from three prior nodes. Physician P suggests/recommends a non-Rx treatment Y, which may include, but not limited to, exercise, dietary restrictions, etc., for patient A based on diagnosis D taking into consideration H, during T,τ. Representationally, Y is a two-dimensional array with y≥1 element(s) since physician P can prescribe multiple regimens with different paradigms. The annotation of derived nodes can be reduced to the atomic elements:

Y→f[P,D,H]→f[P,{D},{H}]→f[P,f[P,R,S,(H)],f[A,T,τ]]→f[P,f[P,{R},{S,(H)}],f[A,T,τ]]→f[P,f[P,f[A,M],f[A,T,τ]],f[A,T,τ]]→f[P,f[P,f[A,{M}],f[A,T,τ]],f[A,T,τ]]→f[P,f[P,f[A,f[P,S,(H)]],f[A,T,τ]],f[A,T,τ]]→f[P,f[P,f[A,f[P,{S,(H)}]],f[A,T,τ]]f[A,T,τ]]→f[P,f[P,f[A,f[P,f[A,T,τ]]],f[A,T,τ]],f[A,T,τ]]→F_{Y}[A⁴,P³,T,τ]

Patient has an overall higher impact on node via diagnosis D and the associated node inheritances that are owned by patient A and lifestyle via history H. This is exhibited through the node-linkages of Y←D←[R]←S(H)←A also Y←H←A.

As illustrated in FIG. 5, physician P owns the derived node notes (N): N→f[P, A, H, S, L, R, D, T, τ], which inherits from multiple prior nodes. Physician (P) owns the notes, the most dynamic record of the patient's health journey, which may evolve across multiple encounters τ, along the time continuum T. Here, each of the pure nodes are included explicitly since it is possible that despite capturing data via inheritances all aspects of the pure nodes might not be conveyed to the node. Similarly, all aspects of the result R might not be explicitly included in the diagnosis D. Test modalities M are not explicitly included since these are incorporated as the origin of the results. Representationally, N is unstructured.

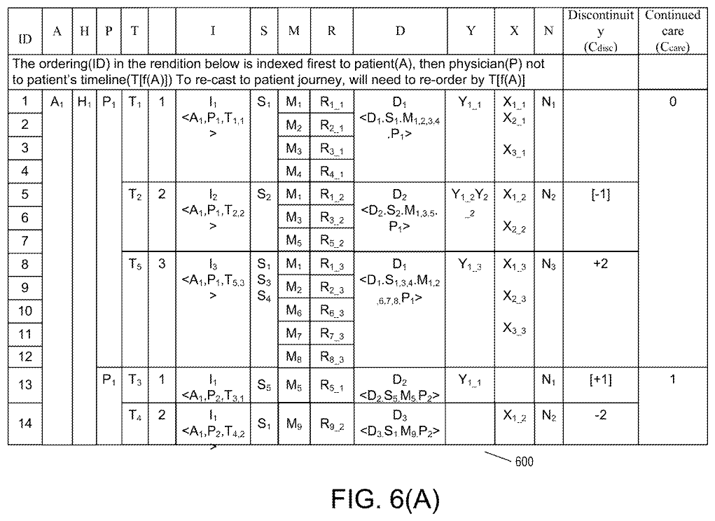

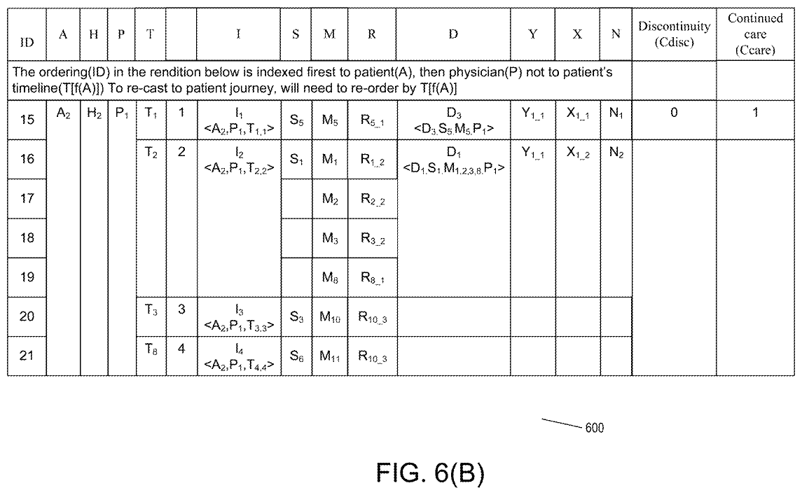

FIGS. 6(A)-(B) illustrate of an example interaction I stratification table 600 for the A×P ecosystem, in accordance with an example implementation. Partial and complete care continuum for a patient is detailed and tracked in the interaction I stratification table. As illustrated in FIGS. 6(A)-(B), care provided can be continuous or episodic. Two patients A₁ and A₂ and two physicians P₁ and P₂ are used in the provided example. Patient A₁ has interactions with both P₁ and P₂: S_{A}_{₁}⊃(A₁×P₁, A₁×P₂). Patient A₂ has interactions with P₂ only: S_{A}_{₂}⊃(A₂×P₁).

Care discontinuity is introduced when a patient transfers across physicians. A patient can continually transfer across different physicians or can transfer out of a physician practice and into a different practice and transfer back into the prior practice. In this mode at least one physician will always treat a patient across a care discontinuity.

Modeling of S_{A1}⊃(A₁×P₁,A₁×P₂) can follow multiple paradigms, some where the effects of discontinuity can be short-lived and some where the effect can be long-lived. For treatment across a discontinuity, a physician will have one of two options: a) treat patient based on knowledge derived from the personal practice only; or b) treat patient based on cumulative knowledge derived from other physicians in addition to self. The former will result in missing data and loss of information for patient status from when patient was under the care of a different physician(s) than current. The latter will reduce information loss by taking into consideration patient status from when patient was under the care of different physician(s) than current.

Paradigm A is where patient is consistently under the care of a single physician with occasional digression S_{A}_{₁}⊃(A₁×P₁, A₁×P₂, A₁×P₁, A₁×P₁, . . . A₁×P₁). In this instance the effect of the discontinuity is short-lived. Here the model tests for equivalence of diagnoses across the discontinuity by comparison of the intra-physician S_{A}_{₁}⊃(A₁×P₁, A₁×P₁, A₁×P₁, . . . A₁×P₁) versus inter-physician S_{A}_{₁}⊃(A₁×P₁, A₁×P₂, A₁×P₁, A₁×P₁, . . . A₁×P₁) interactions.

A slight modification on Paradigm A involves a continuous single patient to single physician interaction mode. In this case, the patient is under the care of the same physician or care coordinated by the same physician. This is represented as S_{A}_{₁}⊃(A₁×P₁, A₁×P₁(A₁×P₂), A₁×P₁, A₁×P₁, . . . A₁×P₁). This illustrates the case where the care may be under a different physician P₂ but is coordinated by the same physician as in P₁—so this illustrates the case where P₁ will have information derived from the care by P₂.

Paradigm B involves shifting of patient care with some non-occasional periodicity between a generalist and specialist. For example, a patient sees a generalist for routine physical examinations then might need to see a specialist such as a cardiac surgeon and might need to return to the same specialist periodically while routine physicals continue to occur with the generalist. This is represented by: S_{A}_{₁}⊃(A₁×P₁, A₁×P₂, A₁×P₁, A₁×P₂, A₁×P₁, A₁×P₁, A₁×P₁, A₁×P₂, A₁×P₁, A₁×P₂, A₁×P₁)

In this instance, the effect of the discontinuity is longer-lived. The patient treatment regimen will be different if the generalist does or does not consider the knowledge derived from the cardiac specialists. Here, the model tests for equivalence of diagnoses across the discontinuity by comparison of the intra-physician (S_{A}_{₁}⊃(A₁×P₁, A₁×P₁, A₁×P₁, A₁×P₁, A₁×P₁, A₁×P₁, A₁×P₁)) versus inter-physician (S_{A}_{₁}⊃(A₁×P₁, A₁×P₂, A₁×P₁, A₁×P₂, A₁×P₁, A₁×P₁, A₁×P₁, A₁×P₂, A₁×P₁, A₁×P₂, A₁×P₁)) interactions. A₁′ represents the exclusion of any interaction involving P₂—in this case the generalist P₁ is not considering inclusion of care across the discontinuity—which is represented by when the patient sees P₂ intermittently.

Paradigm C involves high frequency patient care shifts, for example, military deployments, etc. In this case, the only viable mode of treatment is that of cumulative knowledge across physicians. This can be represented by:

S_{A}_{₁}⊃(A₁×P₁,A₁×P₂,A₁×P₃,A₁×P₃,A₁×P₄,A₁×P₄,A₁×P₅,A₁×P₆)

In the above provided example, P₁-P₆ represent six independent physicians.

A partial care continuums across physician interactions with physicians P₁ and P₂ for patient A₁ is represented in IDs 1-12 and 13-14 of FIGS. 6(A)-(B). IDs 1-12 model S_{A}_{₁}⊃(A₁×P₁) with a discontinuity as represented by T₁, T₂, and T₅. On the other hand, IDs 13-14 model S_{A}_{₁}⊃(A₁×P₂). In this instance, the interaction with one physician, P₂, is continuous but is discontinuous with respect to another physician, P₁.

A complete care continuum for patient A₁ across physician interactions with physicians P₁ and P₂ is represented by IDs 1-14 of FIGS. 6(A)-(B), with S_{A}_{₁}⊃(A₁×P₁,A₁×P₂). When projected to the patient's health journey, the IDs would be rearranged based on timeline T in the sequence of T₁, T₂, T₃, T₄, and T₅ to form I′=I: 1-7, 13, 14, and 8-12. For patient A₂, a complete care continuum is represented by IDs 15-21 as in model S_{A}_{₂}⊃(A₂×P₁).

As illustrated in FIGS. 6(A)-(B), if care is continuous in a given A×P ecosystem, the continued care C_{care} is labeled as 1, otherwise the value is 0. In the example provided:

C_{care}=1: A₁×P₂ and A₂×P₁;

C_{care}=0: A₁×P₁; and

For C_{care}=0, then discontinuity C_{disc} (representing partial care continuum) is established by considering S_{A}_{₁}: By monitoring discontinuity in treatment vis-à-vis physician and indexing with reference to T and bookends −/+: pre/post are labeled. Cardinal values track both number and sequential nature of discontinuity. For example: T₁, T₂[−1], T₃[+1], T₄[−2], and T₅[+2].

The two key features for generation of actionable insights across physician discontinuities to create a complete care continuum for the patient require: 1) modeling of diagnosis; and 2) modeling of treatment—which is analogous to the framework for diagnosis modeling. Additionally, hidden structure discovery is applied to both diagnosis and treatment modeling in prediction generation based on uncovered hidden structure, and will be discussed in detail as part of the technology cascade. Modeling of diagnosis is described in more details below.

### Modeling of Diagnosis

Modeling of diagnosis in the context of a single physician:

Sₚ⊃(A₁×P, A₂×P . . . , Aₙ×P) comprise of multiple paradigms of equivalent diagnoses with differing non-identity of conditions. Under this circumstance, the model training evolves and learns from the same physician treating the same patient along their health time-lines or different patients along their individual health time-lines. This highlights the hidden structure discovery within a given physician's treatment approach. This will also highlight potential errors in a physician's treatment approach based on any adverse outcomes observed within or across patients under their treatment. The modeling of diagnosis will invoke the physician micro-lake, a constituent of the physician lakescape as detailed in the technology cascade section. This also requires the creation and utilization of single physician thesaurus—discussed in details under the hidden structure discovery section.

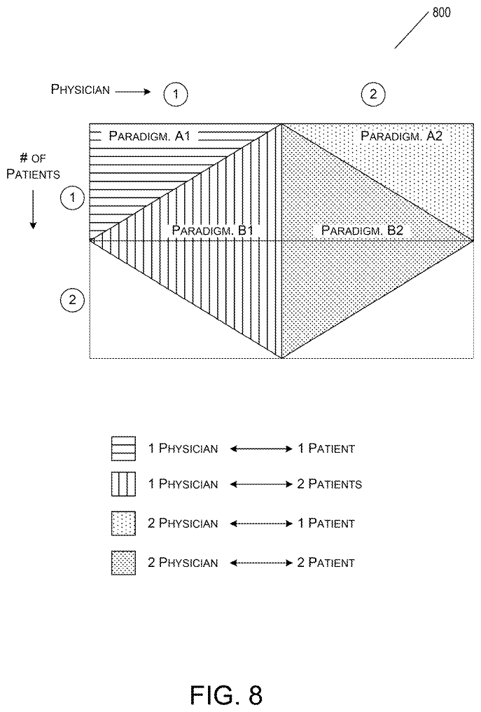

FIG. 8 illustrates example paradigms 800 associated with the modeling of diagnosis, in accordance with an example implementation. As illustrated in FIG. 8, Paradigm A1 involves a case of equivalent diagnosis D=D₁ (D₁∈A₁×P₁) rendered by same physician P for same patient A based on non-identical tests M, symptoms S, and patient history H as a f(ρ). The goal is to discover/establish equivalence in diagnosis for any given physician despite non-identity of conditions, for example, non-identical symptoms, test modalities, etc. Take the following as example, D₁ of pneumonia associated with IDs 1-4: <D₁.S₁.M_{1,2,3,4}.P₁> and IDs 8-12: <D₁.S_{1,3,4}.M_{1,2,6,7,8}.P₁>. To model the equivalence of diagnosis, the flow of information across all nodes at interactions I₁<A₁, P₁,T₁,τ₁> and I₃<A₁,P₁,T₅,τ₃> as described in the data model need to be considered.

The patient history H (H₁, H₃) is evolved over p under Paradigm A1. This requires the transformation from unstructured to structured data using the technology cascade described below to discover the hidden data structure that supports the conclusion of equivalent diagnosis despite non-identity of conditions.

Different symptoms S leading to equivalent diagnosis under Paradigm A1: S1_τ1 @I₁<A₁,P₁,T₁,τ₁> and S_{1,3,4}__τ₃ @I₃<A₁,P₁,T₅,τ₃> are non-identical, but there is direct and/or derived intersection of conditions which enables the establishment of equivalence.

Different test modalities and associated results R leading to equivalent diagnosis under Paradigm A1: M_{1,2,3,4}_τ₁, and M_{1,2,6,7,8}_τ₃ and associated results are non-identical but there is direct and/or derived intersection of conditions which enables the establishment of equivalence.

Different treatment regimen Rx and non-Rx are a consequence of equivalent diagnosis under Paradigm A1: While X: f(X₁_τ₁, . . . X₁_τ₃) and f(Y₁_τ₁, . . . Y₁_τ₃) are different, they are the consequence of the same diagnosis. This implies a hidden connection and/or correlation as deemed by a domain expert—the physician.

The notes N(N₁, N₃) are evolving over p under Paradigm A1. This requires the transformation from unstructured to structured data using the technology cascade described below to discover the hidden data structure that supports the conclusion of equivalent diagnosis despite non-identity of conditions.

Paradigm B1 involves equivalent diagnoses D=D₁ (D₁∈((A₁×P₁)∪(A₂×P₁))) by the same physician P for different patients, A₁ and A₂ based on non-identical tests M and symptoms S, history H as f(ρ(A, P)). The goal is to discover/establish equivalence despite non-identity of conditions. Take the following as example, D₁ of pneumonia associated with IDs 1-4:<D₁.S₁.M_{1,2,3,4}.P₁>, IDs 8-12::<D₁.S_{1,3,4}.M_{1,2,6,7,8}.P₁>, and IDs 16-19: <D₁.S₁.M_{1,2,3,8}.P₁>. To model the equivalence of diagnosis the flow of information across all the nodes at interactions I₁<A₁,P₁,T₁, τ₁>, I₃<A₁,P₁,T₅, τ₃> and I₂<A₂,P₁,T₂, τ₂> as described in the data model need to be considered.

The patients' histories H(f(H₁(A₁)), f(H₂(A₂))) are evolving over ρ₁ and ρ₂ under Paradigm B1. This requires the transformation from unstructured to structured data using the technology cascade described below to discover the hidden data structure that supports the conclusion of equivalent diagnosis despite non-identity of conditions.

Different symptoms S leading to equivalent diagnosis under Paradigm B1: S₁_τ₁ @ I₁<A₁,P₁,T₁,τ₁>, S_{1,3,4}_τ₃ @ I₃<A₁,P₁,T₅,τ₃> and S₁_τ₂ @ I₂<A₂,P₁,T₂,τ₂> are non-identical, but there is direct and/or derived intersection of conditions which enables the establishment of equivalence.

Different test modalities and associated results R leading to equivalent diagnosis under Paradigm B1: Test modalities (M_{1,2,3,4}_τ₁,M_{1,2,6,7,8}_τ₃)f(A₁) and (M_{1,2,3,8}_τ₂)f(A₂)) and associated results are non-identical but there is direct and/or derived intersection of conditions which enables the establishment of equivalence.

Different treatment regimens Rx and non-Rx are a consequence of equivalent diagnosis under Paradigm B1: While Rx: f(X₁_τ₁, . . . X₁_τ₃)f(A₁) and f(X₁_τ₂)f(A₂)) and non-Rx: f(Y₁_τ₁, . . . Y₁_τ₃)f(A₁) and f(Y₁_τ₂)f(A₂)) are different. They are the consequence of the same diagnosis implying a hidden connection and/or correlation as deemed by a domain expert—the physician.

The Notes N(f(N₁, N₃)f(A₁) and (N₂)f(A₂)) are evolving over f(ρ(A,P)) under Paradigm B1. This requires the transformation from unstructured to structured data using the technology cascade described below to discover the hidden data structure that supports the conclusion of equivalent diagnosis despite non-identity of conditions.

Modeling of diagnosis in the context of multiple physicians: Uₖ₌₁^{K}Sₚ_{ₖ}⊃((A₁×P₁,A₂×P₁ . . . , Aₙ×P₁), (A₁×P₂,A₂×P₂ . . . , Aₘ×P₂), . . . (A₁×Pₖ, A₂×Pₖ . . . , Aₘ×Pₖ)) comprises of multiple paradigms of equivalent diagnoses with differing non-identity of conditions. Under this circumstance, the model trains, evolves and learns from the multiple physicians treating the same patient along their health time-lines or different patients along their individual health time-lines. This highlights the hidden structure discovery across the treatment approaches across multiple physicians. This will also highlight potential errors in a physician's treatment approach based on any adverse outcomes observed within or across patients under their treatment and by contrasting the treatment approaches across physicians. The modeling of diagnosis will invoke the physician micro-lake, a constituent of the physician lakescape as detailed in the technology cascade section. This also requires the creation and utilization of multi-physician thesaurus, which will be described in more details below under the hidden structure discovery section. Lastly, it exemplifies the framework for providing the continuum of patient care across physician discontinuities. It is the extension of this framework that will form the core for the differential diagnosis engine that will be deployed to ER/ED scenarios.

Paradigm A2 is a case of equivalent diagnosis D=D₂ (D₂∈((A₁×P₁,I₂) (A₁×P₂,I₁))) by different physicians P₁ and P₂ for same patient A₁ based on non-identical tests M and symptoms S, history H, as a f(ρ(A,Pₚ)). The goal is to discover/establish equivalence despite non-identity of conditions. Take the following as example, D₂ of type II diabetes associated with IDs 5-7: <D₂.S₂.M_{1,3,5}.P₁> and ID 13: <D₂.S₅.M₅.P₂>. To model the equivalence of diagnosis the flow of information across all the nodes at interactions I₂<A₁, P₁, T₂,τ₂>I₃ and I₁<A₁,P₂, T₃,τ₁> as described in the data model need to be considered. The patients' histories H are evolving over f(ρ(A,P)) under Paradigm A2. This requires the transformation from unstructured to structured data using the technology cascade described below to discover the hidden data structure that supports the conclusion of equivalent diagnosis despite non-identity of conditions.

Different symptoms S leading to equivalent diagnosis under Paradigm A2: S₂_τ₂ @I₂<A₁,P₁,T₂,τ₂> and S₅_τ₁ @ I₁<A₁,P₂,T₃,τ₁> are non-identical. Although it involves the same patient it involves two different physicians. However, there is direct and/or derived intersection of conditions which enables the establishment of equivalence.

Different test modalities and associated results R leading to equivalent diagnosis under Paradigm A2: Test modalities (M_{1,3,5}_τ₂)f(P₁), and (M₅_τ₁)f(P₂) and associated results are non-identical but there is direct and/or derived intersection of conditions which enables the establishment of equivalence. In this example, test M₅ is common between two independent physicians, however the test is given to the patient at two different time-points T₂ and T₃ along the patient's timeline.

Different treatment regimen Rx and non-Rx are a consequence of equivalent diagnosis under Paradigm A2: While Rx: f(X₁_τ₂, . . . X₁_τ₂)f(A₁) and f(x:{null})f(A₂) and non-Rx: f(Y₁_τ₁,_τ₂)f(A₁) and f(Y₁_τ₁)f(P₂) are different, they are the consequence of the same diagnosis implying a hidden connection and/or correlation as deemed by a domain experts—the physicians. In this example, the treatments could be very different or near identical. This example is foundational for development alternative medication regimen as a potential way to mitigate shortage or could be highlighting a hidden error in physician treatment paradigm the latter of course would require corroboration by adverse and/or sub-optimal outcome. It would also be foundational in determining a contrast between treatments for patient outcome.

The Notes N(f(N₂)f(P₁) and (N₁)f(P₂)) are evolving over f(ρ(A,P)) under Paradigm A₂. This requires the transformation from unstructured to structured data using the technology cascade described below to discover the hidden data structure that supports the conclusion of equivalent diagnosis despite non-identity of conditions.

Paradigm B2 is a case of equivalent diagnosis D=D₃ (D₃ε((A₁×P₂,I₁) (A₂×P₁,I₁))) by different physicians P₁ and P₂ for different patients A₁ and A₂ based on different tests M manifesting different S with different histories H as f(ρ(A,P)). The goal is to discover/establish equivalence despite non-identity. Take the following as example, D₃ of dehydration associated with ID 14: <D₃.S₁.M₉.P₂> and ID 15: <D₃.S₅.M₅.P₁>. To model the equivalence of diagnosis the flow of information across all the nodes at interactions: I₂<A₁,P₂,T₄,τ₂> and I₁<A₂,P₁,T₁, τ₁> as described in the data model need to be considered.

The patients' histories H are evolving over f(ρ(A,P)) under Paradigm B2. This requires the transformation from unstructured to structured data using the technology cascade described below to discover the hidden data structure that supports the conclusion of equivalent diagnosis despite non-identity of conditions.

Different symptoms S leading to equivalent diagnosis under Paradigm B2: S₁_τ₂ @I₂<A₁,P₂,T₄,τ₂> and S₅_τ₁ @ I₁<A₂,P₂,T₂,τ₁> are non-identical conditions involving different physicians and different patients. However, there is direct and/or derived intersection of conditions which enables the establishment of equivalence.

Different test modalities and associated results R leading to equivalent diagnosis under Paradigm B2: Test modalities (M₉_τ₂)f(A₁,P₂), and (M₅_τ₁) (A₂,P₁), and associated results are non-identical but there is direct and/or derived intersection of conditions which enables the establishment of equivalence.

Different treatment regimen Rx and non-Rx are a consequence of equivalent diagnosis under Paradigm B2: While Rx: f(x₁_τ₂)f(A₁, P₂), and f(x₁_τ₁)f(A₂,P₁)) and non-Rx: f({null})f(A₁, P₂), and f(y₁_τ₁)f(A₂,P₁)); are different, they are the consequence of the same diagnosis implying a hidden connection and/or correlation as deemed by domain experts—the physicians. In this example, the treatments could be very different or near identical. This example is foundational for development alternative medication regimen as a potential way to mitigate shortage or could be highlighting a hidden error in physician treatment paradigm the latter of course would require corroboration by adverse and/or sub-optimal outcome. It would also be foundational in determining a contrast between treatments for patient outcome.

The notes N(f(N₂)f(A₁, P₂) and (N₁)f(A₂, P₁)) are evolving over f(ρ(A,P)) under Paradigm A2. This requires the transformation from unstructured to structured data using the technology cascade described below to discover the hidden data structure that supports the conclusion of equivalent diagnosis despite non-identity of conditions.

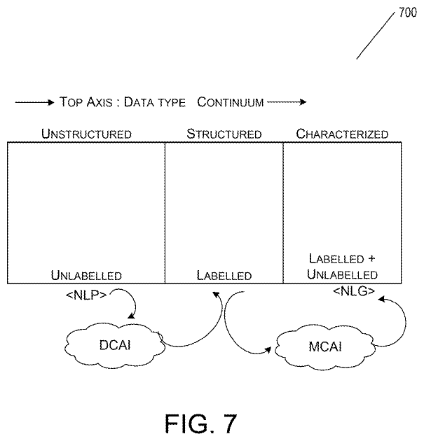

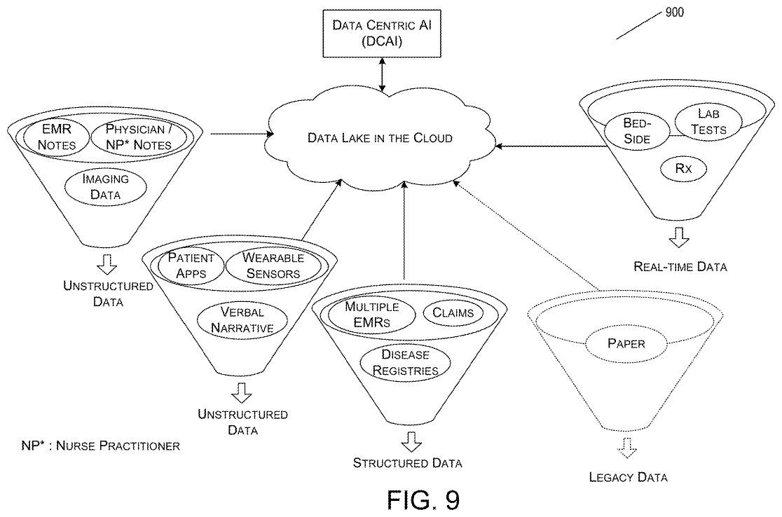

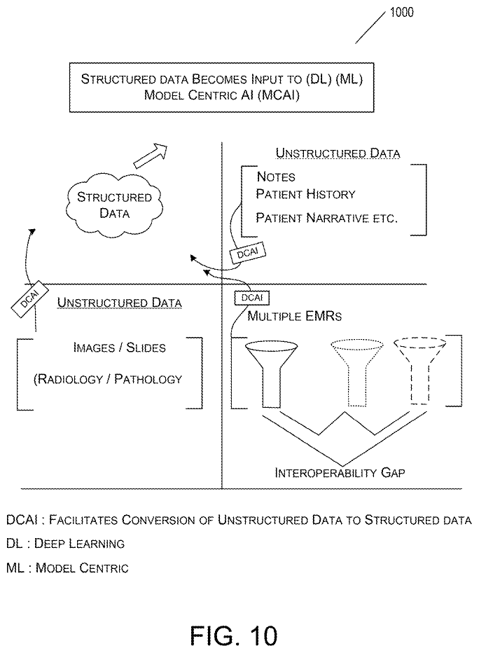

FIG. 9 illustrates an example ecosystem 900 of healthcare data, in accordance with an example implementation. The eco-system of healthcare data comprises of approximately 20% structured or labeled data and the rest 80% is unstructured or unlabeled. FIG. 7 illustrates an example data structuring process 700 using unstructured data, in accordance with an example implementation. The unstructured data comprise primarily of notes originating from physicians, nurse-practitioners, other medical personnel, as well as patients themselves inclusive of their families. The unstructured data source can also comprise of images of different origin such as radiology, pathology among others. All ML/AI based engines, referred to as model-based AI(MCAI), require labeled or structured data as input. FIG. 10 illustrates example data inputs 1000 for a model-centric AI, in accordance with an example implementation. Therefore, to utilize most healthcare data the transformation from unstructured to structured is key. The detail of this process is described in the technology cascade.

There is a diversity of data elements in the current healthcare ecosystem. The data elements are modeled as a function of type, streaming and update frequency (UF). Data types are structured (S) or unstructured (U). Data streaming is real-time(R) or batched (B). Real-time (R) refers to the mode where the data is extracted from the data source and ingested as soon as it is generated. This is a continuous flow of the data, and the frequency of update is NULL. Batched (B) refers to the mode where the data is extracted from the data source and ingested in discrete chunks after it has been generated. This is a discontinuous flow of the data. It can be frequent, for example, every 10 minutes, 1-hour, etc., or infrequent updates, for example weekly, monthly, etc. For batched, frequency of update will be designated. In some example implementations, the update frequency is determined and set by a user/operator.

The data elements in the current healthcare ecosystem are the following: claims, notes, laboratory test results of Labs, electronic medical/health records, Electronic Medical (Health) Record (EM(H)R), bedside data in the ER or bed-data, legacy paper records, and other elements comprising of clinical trials and registries. The data elements are described in details below.

Claims—this comprises of an admixture of structured and unstructured data types but enriched for the former. This is not available in real time and is time-delayed by months in terms of its content.

Claims→f(Claims-Matrix{S,U},B,UF=3 months)

Where enrichment is represented via bold lettering. Enrichment implies that the majority of the data (as in >50%) is structured in nature.

Claims data convey differential weights for the patient and physician. With regards to patient, claims data is incomplete. It attempts to synthesize a treatment/medication suggested but not the effect or the outcome. It also does not reflect the true adherence of the patient to the Rx treatment suggested by the physician. The weight associated with claims data from a patient centric model is LOW to MODERATE. With regards to the physician, claims data can identify the pattern(s) of physician treatment. The weight associated with claims data from a patient centric model is HIGH.

Notes—notes can originate from multiple sources and primarily comprise of unstructured data. Notes origin governs the degree of structure in the data. Notes from physicians and nurse practitioners are enriched for unstructured data since these usually originate from domain experts who might consider expressing their opinions outside of the bounds of structure provided by the constraints of EMR, etc. Notes from clinic staff are enriched for structured data by way of using established controlled vocabulary, such as pull-down menu items since domain expertise is not readily available here. Lastly notes from EMR/EHR: comprise of structured using established controlled vocabulary, as well as unstructured data by way of embedded notes.
Notes→f(Notes—Matrix{S,U},B,UF=coincidental with patient visit both pre and post)

Labs—This is a matrix of multiple sub-elements such as allergies, immunizations, specialized tests which are enriched for structured data, and imaging which is enriched for unstructured data.

Labs Allergies—This is modeled as a function of allergy (type), allergen (if known), methodology used to establish the allergy, and date of the event. The data type is enriched for being structured data. Allergy tests and results are not available in real-time.

Allergies→f(Allergy-Matrix{S,U},B,UF=coincidental with patient visit)

Allergy-Matrix→f(allergy-type→Matrix([Allergen Method Date])

Allergies: Data is segmented under seven major categories as per the guideline of the asthma and allergy foundation of America: drug, food, insect, latex, mold, pet, and pollen. There is an additional category of ‘other’, which is a catch-all for entries that cannot be categorized directly under the seven categories above. The method or methodology used to establish an allergy is either a skin test which is positive or a challenge such as anaphylaxis.

Allergies Matrix is defined as the following:

| | Entity/ | Test: (type, | Challenge: | Date of |
|---|---|---|---|---|
| | Allergen | outcome) | Description | Event |
| Drugs → | NSAID | NULL | Anaphylaxis | Feb. 2, 2022 |
| n × n Matrix | Drug-X | | | |
| Food → | Almond | (Skin, 1) | NULL | Feb. 2, 2002 |
| n × n Matrix | Food-X | | | |
| Insect → | Bee Sting | NULL | Anaphylaxis | Aug. 9, 2012 |
| n × n Matrix | Insect-X | | | |
| Pets → | Cat | (Skin, 1) | Sneezing | Feb. 2, 2002 |
| n × n Matrix | Dander | (Skin, 1) | | Dec. 12, 2012 |
| | Pet-X | | | |
| Latex → | | | | |
| 1 × n Matrix | | | | |
| Mold → | | | | |
| 1 × n Matrix | | | | |
| Others | Structured or | | | |
| | Unstructured | | | |

1. Drug: An example of a common drug allergy is penicillin as established via a positive skin test. Another example is via a challenge such as anaphylaxis to non-steroidal anti-inflammatory drugs (NSAID). No allergies to known drugs are generally designated as no allergies to known drugs (NAKD) and the associated test or challenge is a NULL.
2. Food: Food allergies can be specific or non-specific and consequently captured in either structured or unstructured form. Allergens for example, almond, peanut, and nut flavor as established via a positive skin test. Nut flavor is very non-specific allergen in terms of real-world food consumption.
3. Pets: This can be specific or non-specific and captured either by a structured or unstructured data element. An example of non-specific is cat/feline products. If the allergen is specifically annotated as ‘dander’, then the patient could be allergic to both cats and dogs for example.
4. Environmental: Allergies can be specific or non-specific and consequently captured in either structured or unstructured form. An example of a structured element can be specific allergens such as grass, pollen, etc. This however can be annotated in EMRs under a non-specific category such as ‘other’.

Labs Immunizations—This is modeled as a function—main immunization category, subcategory highlighting the delivery mechanism and the data associated with the event. The data is enriched for being structured and should be available in real-time.
Immunization→f(Immunization-Matrix{S,U},B,UF=coincidental with visit)
Immunization-Matrix→f(immunization-type→Matrix ([MainCategory SubCategory DateArray])
An example is influenza vaccine with subcategories nasal and whole—both of which are not synthesized under the influenza main category. There are also cases where the data for influenza whole is redundantly present under Influenza unspecified. This is an example of an aspect of the data harmonization (described below) necessary in reconciliation of EMR data.
   Example: where influenza vaccines are given on the following dates: Nov. 15, 2021, Nov. 24, 2020, Feb. 22, 2020, Oct. 10, 2018, Nov. 3, 2017, Nov. 13, 2014, Oct. 10, 2012, Nov. 10, 2010, Oct. 17, 2009
   The subcategories:
      Influenza nasal: Nov. 16, 2011, Nov. 10, 2010;
      Influenza whole: Sep. 30, 2015; and
      Influenza unspecified: Sep. 30, 2015.

Labs Test—This is a matrix of routine and specialized tests and can be structured or unstructured and might or might not be available in real-time. This is modeled as a function of 5 primary components: test modality, testing location, results, normal range, and value specific to the patient. The model supports assimilation of results via both supervised and unsupervised learning.
Tests→f(Test-Matrix{S,U},B/R,UF=coincidental with/post patient visit)
Test-Matrix→f(immunization-type)→Matrix
([Modality Location: Home/Clinic ResultsArray])
ResultsArray→Matrix([Value Normal: Low Normal: High])
Examples of modality: Complete Blood count (CBC) test, Lipid Panel, etc.
   Liquid Biopsy tests
Examples of at home tests: COVID 19, Sleep Apnea, etc.
Examples of specialized tests using a controlled vocabulary:
   Liquid Biopsy tests: Galleri (GRAIL), Prospera (Natera), etc.
Examples of specialized tests without using a controlled vocabulary:
   SNPs, Transcriptomics, Proteomics, etc.

Labs Imaging—Images can be digitized in PACS and/or EMR or might be non-digital as in film. Pathology images can be digitized and/or might exist as pathological reports in EMRs or stored locally within the physicians' practices. These are all unstructured and the digitized images are usually not real-time since they need to be reviewed by experts. The images might be coupled with structured and/or unstructured notes.
Images→f({U},B/R,UF=coincidental with/post patient visit)

Patient generated data (PGD)—Patient generated data can be both unstructured and structured. Traditional data such as audio, video, email, and text messages are unstructured, not available in real time and might be archived. Narratives by patients and their families constitute a significant portion of the patient health history, are entirely unstructured and are generated in real-time. Data can also be derived from Internet of Medical Things (IoMT) such as patient apps on smart devices, wearables, bio-sensors, virtual and augmented reality devices. These are structured data, potentially available in real-time, could be batched.

EM(H)R—The structured data do usually adhere to fast healthcare interoperability resources (FHIR) standards and access is batched. Even within structured data, there are interoperability constraints across different EMR systems (vendors) or multiple instances for the same EMR system There are also notes, emails, text, and other unstructured data.

Bed data—This is data that is from instruments connected to the bed, laboratories test results while the patient is in the ER, as well as medication and diet-related data. These are primarily structured data, available in real-time. This data is high volume and are not captured in detail in the EMR.

Legacy paper records are also available for individual patients and would use OCR for transcription to unstructured and transform the unstructured data into structured data.

The last data element involves outcomes from clinical trials, registries and literature. These are at the level of patient cohorts as opposed to individuals. All the prior data elements are available at an individual level.

The technology cascade supporting the platform in the performance of autonomous medical operation is modeled as stackable bricks. There are seven bricks in a sequential segment mapped to the colors VIBGYOR—rainbow. The functionality of each brick is mutually exclusive. Any brick can be optimized independently from one another. The bricks can be connected to one another and excluded provided the VIBGYOR sequence is maintained. For example if the bricks G and O in the technology cascade are not invoked, they will then be designated as null using the following annotation: VIBG{null}YO{null}R. Each brick can be developed as a tower. Each brick can be represented as a whole or partial brick depending on the intensity or strength of the touchpoints of each node (described above) at a given brick.
Brick V (Violet): Data Ingestion
Brick I (Indigo): Data Management
Brick B (Blue): Metadata Creation
Brick G (Green): Data Centric AI (DCAI)
Brick Y (Yellow): Structured Data Merge
Brick O (Orange): Model Centric AI (MCAI)
Brick R (Red): Insights Generation

In some example implementations, bricks V and R are invoked only once during the execution of the insight generation pipeline. Specifically, data elements are ingested only once at the very onset and insights are generated at the very end of the process.

Nodes described in the A×P model invoke bricks in the technology cascade in the sequence specified by VIBGYOR. For example, brick Y cannot be invoked before brick B.

Each node in the A×P model will have either major, minor, or null touch with the bricks in the technology cascade. Major touchpoints are indicated by significant intensity and impact. Minor touchpoints are indicated by nominal effect. Null touchpoints occur when any brick in the technology cascade is excluded in a node.

The intensity at the touchpoints will be governed by the content of the data elements in each node. For example, if the data elements in a node enrich for unstructured then high complexity transformational processes will need to occur to generate high fidelity structured data from them, involving a major touchpoint.

Brick V: This brick constitutes the data ingestion engine. Data elements which originate from the current care paradigm have been described above. Brick V will store all data elements in a data lake (DL). A DL is critical to support the storage of all data elements within a single infrastructure. It also makes the framework extensible to future formats. Brick V ingests all data present in the ecosystem where the platform is deployed and first create an immutable data store in the DL. There will be no transformation upon loading into the lake.

The data ingestion mechanism is scalable when it comes to ingestion of existing healthcare data sources or data elements. The scalability and extensibility are afforded by the use of a data lake, which is agnostic to data types and structures, and can contain any kind of object within a single repository. The details of data ingestion are described in technology cascade below.

Brick V is flexible to create the DL in a cloud native storage, solid-state drives (SSDs) or hard disk drives (HDDs). The storage can exist behind a designated firewall. The DL can exist in the client's ecosystem where they are the only ones privileged to PHI-access. Brick V processes both structured and unstructured datatypes.

Brick V extracts from source and load to destination repository as a batch and/or in real-time. Infrastructure for real-time data ingestion will utilize technologies such as KAFKA, KINESIS, PUBSUB, etc. Brick V is agnostic to location of source data—it can extract from public cloud (S3 buckets, AZURE blobs, etc.), on-premises, networked edge storage, or any combination of the above. As an example, JDBC/ODBC pipes will be used to access the data from databases resident in the cloud, core, or edge. Brick V supports a federated data store.

Brick I: This brick constitutes the data management engine and tracks the origin and passage of each data element ingested into the DL. Brick I is a tower where multiple bricks are stacked in the following sequence:
I.1) Data provenance;
I.2) Data governance; and
I.3) Data security.

Brick I.1 ensures data provenance. Prior to any data transformation and or use—the origin of the data will be confirmed. The brick embodies the following core functionality. Appropriate labels will designate cloud versus local or core versus edge origin of each data element. Provenance will establish if the data elements were transformed post creation. Provenance will rank-order the data elements based on verification of the origin. The rank-ordering can be used to weigh the influence of the data element in the subsequent steps of DCAI and/or MCAI (described below) for insights generation.

Brick I.2 ensure that data governance in the cloud complies with General Data Protection Regulation (GDPR), Health Insurance Portability and Accountability Act (HIPAA), etc. Governance will not be an explicit part of the platform but will be incorporated via a native framework such as GOOGLE Cloud, SNOWFLAKE, etc., used as the IaaS (Infrastructure as a Service).

Brick I.3 ensures that security in the cloud complies with Service Organization Control Type 2 (SOC2), Health Information Trust Alliance (HITRUST), etc. In some example implementations, cyber security is incorporated via a native framework such as GOOGLE Cloud, SNOWFLAKE, etc., used as the IaaS (Infrastructure as a Service).

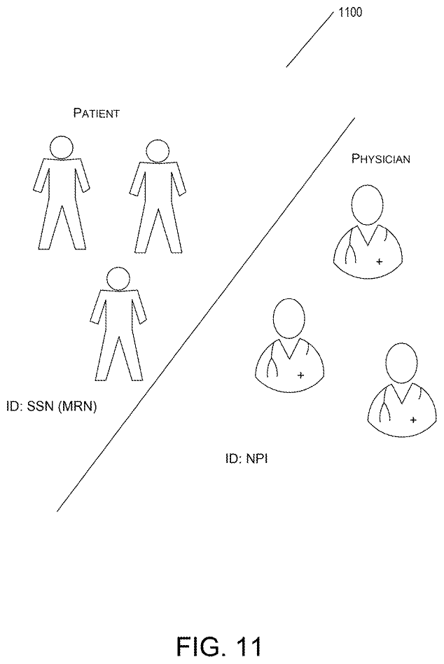

Brick B: This brick constitutes the metadata creation engine which involves linkage creation across data elements in DL, by tagging at multiple levels. This brick is foundational for creating the A×P ecosystem. Brick B initially tags data to identify and segregate patients A and physicians P. Brick B tags patients, A, uniquely, via social security number (SSN) and/or medical record number (MRN) if all are within the same clinic. Brick B tags physicians, P, uniquely via physician identifiers such as National Provider Identifier (NPI). The NPI is a HIPAA-required numerical identifier uniquely assigned to physicians and other healthcare providers. FIG. 11 illustrates tagging process 1100 of patients and physicians using Brick B.

Brick B creates patient lakescape (DL_{A}) and physician lakescape (DL_{P}) within parent DL. DL_{A} and DL_{P} co-exist within DL with identical data but are segmented differently. DL_{A} partitions the data in a patient-centric mode. DL_{P} partitions the data in a physician-centric mode. Each lakescape is a cluster of micro-lakes.

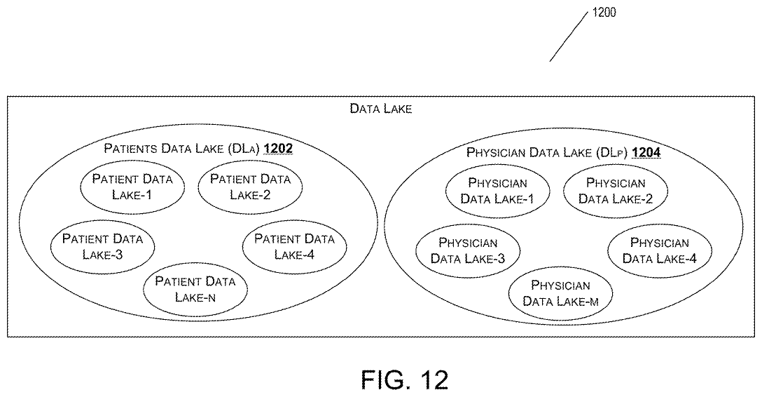

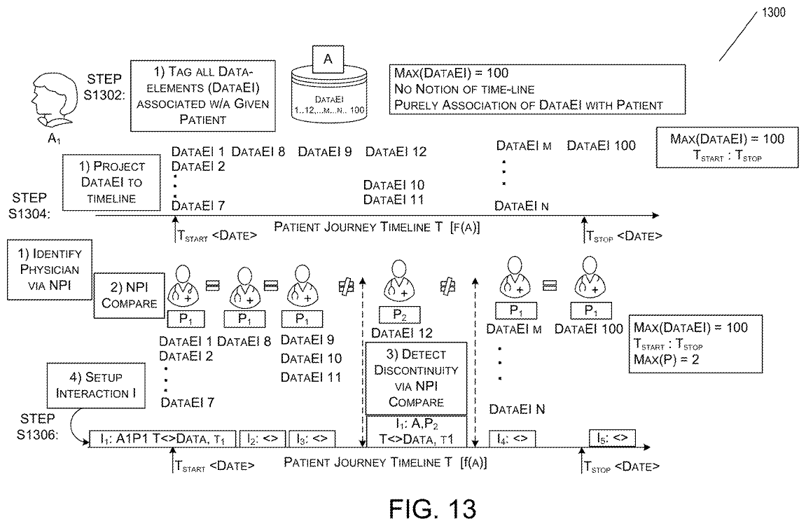

FIG. 12 illustrates an example data lake 1200 with patient-centric micro-lakes and physician-centric micro-lakes, in accordance with an example implementation. Brick B creates a cluster of patient micro-lakes (MLA), which are the constituents of DL_{A} 1202. A linkage between all data elements associated with a unique patient will constitute a unique micro-lake. A cloud-based data-lake creation is represented by the topmost section of FIG. 9. FIG. 13 illustrates an example patient-centric micro-lake generation approach 1300, in accordance with an example implementation. At step S1302, data elements associated with a given patient are tagged. At this point the micro-lakes are a collection of data elements associated with a given patient, but the patient journey timelines have not been created, in other words the data elements associated with a given patient have not been projected to their timeline:

DL_{A}⊃(MLA₁,MLA₂ . . . ,MLA_{α})

Next, Brick B will create the universe of patient interactions along their timeline through data projection at step S1304. It will do so via identification of the time associated with each data element in each patient micro-lake and create a chronological linkage of data element. This sets up the A×P ecosystem described above, for each patient in DL_{A}. 1202, thereby creating the foundation of the patient health journey platform. The A×P ecosystem comprise over a sequence of interactions that evolve under ρ: I_{ρ}→f[A_{ρ},P_{ρ},(ρ)] at step S1306, and these interactions can encompass more than one physician.

MLA→S_{A}⊃(A×P₁,A×P₂ . . . ,A×Pₙ), where A: constant and P: variable; Therefore, DL_{A}⊃(S_{A}_{₁},S_{A}_{₂}. . . S_{A}_{₃})

The dimensionality of the patient lakescape, DL_{A} 1202 created by Brick B is flexible and scalable. The number of micro-lakes is definable based on the location of the deployment of the platform. For example: it can include all patients across all physicians in a hospital or only all patients across any subset of physicians, where the minimum number is one.

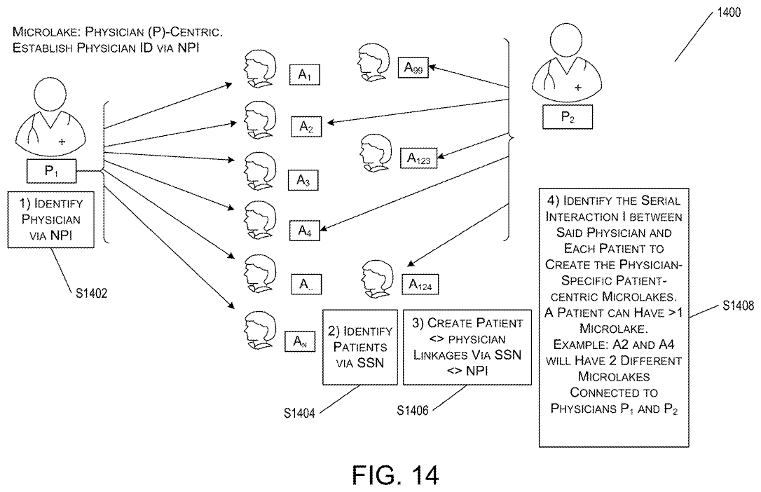

Brick B will then create a cluster of physician micro-lakes (MLP), which are the constituents of DL_{P} 1204. FIG. 14 illustrates an example physician-centric micro-lake generation approach 1400, in accordance with an example implementation. At step S1402, each physician is identified via NPI. Each physician entity (Pₚ) with a unique NPI number is a physician and will be associated with their unique micro-lake (MLPₚ). DL_{P}⊃(MLP₁, MLP₂ . . . , MLPₚ).

Brick B will then create a linkage between all unique physician micro-lakes and their respective patients. The model supports the one to many, patient to physician interaction linkages. Patients are identified via SSN at step S1404. Physician to patient linkage will be established via NPI to SSN (or MRN) association at step S1406.

MLP→Sₚ⊃(A₁×P,A₂×P . . . ,Aₙ×P), where A: variable and P: constant.

Brick B will support a flexible and scale-able dimensionality of the DL_{P} 1204 lakescape. The number of micro-lakes is definable based on the location of the deployment of the platform. For example, it can include all physicians in a hospital or a subset in a specialty practice, or physicians across multiple organizations.

Brick B will support redundancies since the same patient might be linked to multiple physicians. This is exemplified in IDs 1-14; where for patient A₁, IDs 1-12 are attributable to physician P₁ and IDs 13-14 are attributable to P₂.

Brick B establishes linkages between the patient's timeline in the patient lakescape with the physicians' interaction in the physician lakescape at step S1408. This continues to develop the patient's health journey platform.

Brick B establishes linkages between each physician in the physician lakescape to all patients they have interacted with. The interactions are projected to the physician's timeline. In this linkage mode the patient micro-lakes are segmented into sub-micro-lakes, where each sub-micro-lake is specific to the cumulative set of interactions for a unique patient-physician linkage and, therefore, is reflective of a given physician's decision process.

Upon the completion of all linkage establishments, both lakescapes DL_{A} 1202 and DL_{P} 1204 will support a server-side encryption model using for example, AMAZON Web Services (AWS) Key Management service. This will enable the deployment of the subsequent bricks of the technology cascade on de-identified patients and physicians. This also ensures the data is encrypted whenever it is at rest.

Brick G: This brick constitutes the Data Centric AI (DCAI) engine. The roadblock for improved accuracy in clinical insights generation from AI models is access to (or lack of) domain expertise/knowledge. Domain experts can also convey a confirmation bias which will carry onto machine learning models unless determined. A way to establish this bias is a comparison of physicians' decision processes. DL_{P}⊃(MLP₁, MLP₂ . . . , MLPₚ) where there are patient redundancies in a framework that can expose using this platform. Finally, it is not necessarily the sophistication of ML models and or model (software) development, but data management especially of model training data and operationalizing transformation of unstructured data that is essential for generation of augmented intelligence.

Brick G enables extraction of maximal value from unstructured data by transforming them to structured data to be used for training ML models. This brick creates the input for the model centric AI engine (MCAI) described below.

Brick G is a tower where multiple bricks are stacked in the following sequence:
Brick G.1: Unstructured data transformation;
Brick G.2: Data cleaning and labeling;
Brick G.3: Data harmonization;
Brick G.4: Training data management; and
Brick G.5: Hidden data structure discovery.

Brick G.1: This sub-brick powers the unstructured data transformation engine which will use Neuro symbolic AI (NSAI)—a hybrid between Deep learning (DL) and symbolic models—to transform unlabeled data such as notes (N), history (H), images, videos, etc. In NSAI the task of feature extraction is performed by the DL models, this is followed by the manipulation of the features by symbolic approaches. Neuro symbolic concept learner is resilient with small training dataset and will be deployed to retrain or relearn from new data obtained periodically. For example, in the same hospital setting but would now include cohorts of long COVID patients as it is becoming more common-place in the post pandemic era. NSAI model characterization can be implemented via Area Under the Receiver Operating Characteristic (AUROC) analysis, for example.

The NSAI approach will support a sub-model architecture since a simple NLP based approach is insufficient for discovery of nuances. Examples of the NSAI approach implemented in this platform includes sub-models for image and video frame parsing, question parsing and learning from both text and images. Examples of the sub-models are below.
Sub-model for image/video frame parsing: Convolutional Neural Net (CNN);
Sub-model for question parsing: NLP such as Bidirectional Encoder Representations from Transformers (BERT) and its variants, GPT3; and
Sub-model for deriving learnings: Recurrent Neural Net (RNN).

The NSAI approach-based learning will not only summarize the information that is available in the unstructured sources, but can also potentially pose new questions that have not been asked to derive certain hypotheses. This framework will identify missing links in derivation of hypotheses or conclusions. This is particularly instrumental for medical caregivers—physicians and nurses in an ER/ED where they must make very quick decisions often without much time to reflect. This will allow exposure of a potential diagnostic or diagnoses momentum that is the root cause of an anchoring bias—types of confirmation biases without exhaustive/focused exploration of root cause of symptoms.

Brick G.2: This sub-brick powers the data cleaning and labeling engine. Data cleaning, labeling, re-cleaning, and re-labeling are iteratively performed in the absence of supervised domain expertise input.

Brick G.2 enables the dis-ambiguation and de-convolution of epistemic uncertainty from aleatoric uncertainty. Epistemic uncertainty refers to model uncertainty due to lack of training data, underrepresented minority class, non-inclusion, or incomplete definition of an all-comers population, etc. Aleatoric uncertainty refers to uncertainty due to label errors.

Brick G.2 uses heuristics specific to the domain to incorporate weak supervised learning, for example. The goal here would be to solve the problem as one of classification. In the absence of direct access to domain experts—EMR/HER are considered as surrogates for learning the domain specific nomenclature and concepts, direct and/or derived. As a seed, EPIC EMR specific phrases are used to facilitate programmatic data annotation. Given the lack of interoperability across EMRs, the ecosystem of EMR specific nomenclature is developed by considering the union of annotation across EMRs. The superset of annotation will undergo data cleansing using methodologies described below.

Brick G.2 enables the finding and fixing of EMR annotation by discovery of ontological issues, some examples are shown below:
Is-a relationship redundancy labels (CBC→Blood count);
Similarity redundancy labels (UNK→Unknown);
Misnomers—a generic description of a medical condition not reflective of histopathological findings;
Homonyms—multiple definitions of words can be cleansed in a context/scenario dependent manner; and
De-duplication of data.

Brick G.2 enables the cross-referencing of data from notes with structured derivatives in the EMR, achieved via pattern recognition of structured concepts in unstructured data using fuzzy logic.

Brick G.2 uses fuzzy logic-based AI approaches to determine confidence intervals (CI) about concepts being transformed from unstructured to structured. In the estimation of the CI, it will have the option of incorporating the rank-weights introduced by Brick I based on data management.

Brick G.3: This sub-brick powers the data harmonization engine which will harmonize or normalize the data element in each of the nodes in the A×P model and will do so in the context of synthesizing patient data elements across multiple sources across the time-period desired.

Brick G.3 ensures the implementation of a controlled vocabulary (CV). The CV matrix will have six independent concepts CV_{S}, CV_{L}, CV_{M}, CV_{D}, CV_{X}, and CV_{Y}, associated with nodes S, L, M, D, X, and Y. There will be an additional dependent concept CV_{M_units} which will develop in concert with CV_{M}, where the standard units of measurement associated with a given test modality will be established. At the very onset of the pipeline, null entries are set for each of the nodes. Referring back to FIGS. 6(A)-(B), nodes having first word encounter are identified, for example, S₁, S₂, M₁, D₁, etc. In some example implementations, identification can be made in the form of color highlights or color coding to distinguish different types of identifications. First word encounters are appended to the respective CV, for example: CV_{S}={S₁, S₂ . . . }. Successive word encounters are compared against the respective CV. Data-normalization principles enable one of the following two outcomes:
1. Old word (encountered before/recognized): collapse to CV and note action of collapsing in data provenance. Example: “not-known” or UNKNOWN where UNKNOWN already exists in CV_{S}; and
2. New word—add to CV. Example: “Penicillin” to CV_{L}.

Brick G.3 harmonizes inconsistency of units for results from tests across multiple sources for a given SA in the patient lakescape. This will invoke the CV_{M_units}. The harmonization might involve data scaling—for example, Creatinine has units such as Mg/dL, Mmol/L, Mg/L while all are correct, the values from multiple sources should be normalized to the same unit. Assuming the standard unit is Mg/dL, the conversion will be 1, 11.312 and 0.1 respectively. The harmonization might involve standardization of nomenclature—for example, Mg/dl and MG/DL and mG/deciliter all to Mg/dL.

Data harmonization engine performs outlier detection and harmonize values out of normal range for results (R) in the context of the test modality (M) and diagnosis (D). For example, there can be scaling errors that might have the value register blood pressure as 1700/80 mmHg instead of 120/80 mmHg. The value would be re-scaled to 120/80 mmHg provided there is no diagnosis context of hypertension, stroke, heart attack and/or symptoms indicating heart disease. This underscores the connection of the nodes R as measured from M in the context of S/H and D as well as medication and non-medication treatments.

Brick G.4: This sub-brick powers the training data management engine. The goal here is to create a data management framework that maximizes data diversity. This is performed by obtaining world data, and where real-world data is limited, performing noise simulation and/or data extrapolation based on available real-world data through application of discriminative and generative frameworks. In so doing, the emphasis shifts from model-centric optimization to data-centric optimization. This enables adoption of the paradigm that training data is the new code. Therefore, it is critical to invoke brick I prior to this sequence, such that optimal training data can be made available for DCAI model training. MCAI performance (described below) with model optimization saturates fast with not necessarily an effective CDSS.

Brick G.4 reduces training costs—use of GPU—by reducing the need for significant computational power to train the model. Through reduction of model performance, DCAI enables reduction of technical debt.

Brick G.5: This sub-brick powers the hidden data structure discovery engine. Frameworks that constitute the core engine for hidden structure discovery and diagnosis modeling revolve around identification of domain knowledge. The application of the core medical knowledge varies across physicians based on training, years of experience, etc. This critical knowledge is hiding in Notes (N), which account for majority of the unstructured healthcare data.

For Brick G.5 to power hidden data structure discovery, it needs to have both a lexical and morphological awareness. The thesauri described below creates the lexical awareness. Morphological awareness has a significant impact on the interpretation of the vocabulary and/or statement.

To initiate hidden structure discovery, brick G.5 formalizes the creation of two independent thesauri. These are 1) physician-specific thesaurus; and 2) multi-physician thesaurus. A physician-specific thesaurus is created utilizing the CV framework described under brick G.3. The physician specific thesaurus allows the creation of a personalized prediction of each physician's own treatment paradigm. Generation of the physician-specific thesaurus provides operational efficiency as well as ensuring creation of the diagnosis differential in an unbiased manner by the AI algorithm going through all of the physician's treatment history.

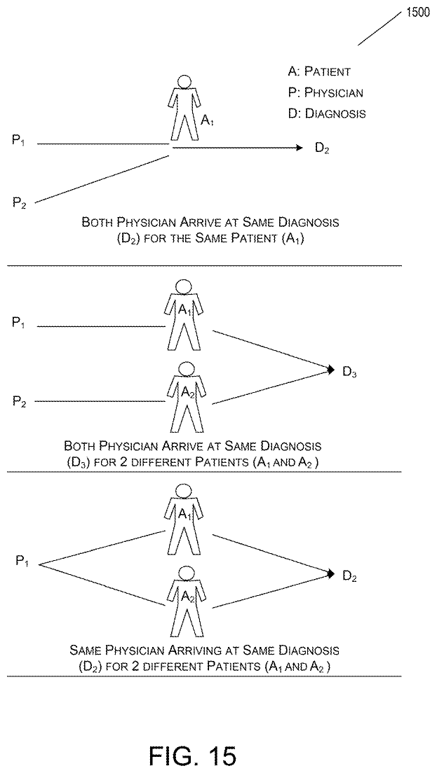

Creation of a multi-physician thesaurus also utilizes the core principle for a physician-specific thesaurus except in the multi-physician context. FIG. 15 illustrates example models 1500 of multi-physician diagnosis thesaurus, in accordance with an example implementation. The multi-physician thesaurus incorporates input from multiple physicians where there might/might not be an intersecting set of patients. The goal of multi-physician thesaurus generation is to discover/establish equivalence in the various nodes across multiple physicians based on a common patient or a set of patients.

To develop and posit an interpretation of a statement or word, brick G.5 incorporates morphological awareness. This awareness is particularly critical in ER/ED where short-hand annotation can often be used. For example: the word ‘unreadable’ comprise of the root word ‘read’ together with the word ‘un’ preceding the root and indicating a ‘negation effect’ and ‘able’ a word following the root indicating the ‘ability to perform or complete a task’.

Brick G.5 establishes equivalence within the context of each individual node under the multi-physician scenario where there is a redundancy or overlap of patients. This invokes the physician lakescape without the chronological linkages that unify the nodes.

∪ₖ₌₁^{K}Sₚ_{ₖ}⊃((A₁×P₁,A₂×P₁ . . . ,Aₙ×P₁),(A₁×P₂,A₂×P₂ . . . ,Aₘ×P₂), . . . (A₁×Pₖ,A₂×Pₖ . . . ,Aₘ×Pₖ))

Brick G.5 implements similarity engines, an embodiment of which might include sentence transformers, such as cosine similarity, to determine concept and or sentence similarities. As the various CVs evolve, the sematic search approaches can be employed specifically to each node. Lexical search such as ElasticSearch, unsupervised learning approaches for example, k-means, agglomerative clustering may be utilized. In some example implementations, Natural Language Inference (NLI) is adopted to help determine/disambiguate if two concepts/hypothesis, as derived through physician-specific thesaurus and multi-physician thesauri, conform/neutral or are in contradiction. For example, “UNK” and “un-known” are neutral vocabularies, as they indicate one and the same concept. On the other hand, “UN-AC” could be un-acknowledged or unaccounted.

Comparison between physician-specific thesaurus and multi-physician thesauri is effective when a patient sees multiple physicians. Each physician may have a different way of deriving diagnosis differential. This allows for a comprehensive decision making as well as spotting differences in opinions between multiple physicians. These differences in opinions might be semantics only but pointing to the same underlying diagnosis. Where opinions significantly differ from one another, this may indicate biases and/or occurrence of errors in a given physician's diagnosis. For example, if a voting scheme is employed and four out of five physicians conclude on a first diagnosis and the remaining physician concludes otherwise, this bias can be potentially estimated by modelling the data in the physician-specific thesaurus.

Brick Y is the Structured data merge engine that enables the merge of the structured data elements along a timeline. It operates in parallel within each of the micro-lakes in each of the lakescapes. The input to this engine is the structured or labeled data elements which are the output from the DCAI engine—Brick G. The output from this brick will be the following on a per micro lake basis:
Patient health journey as projected to a timeline; and
Physician practice pattern data as projected to a timeline.

Brick O powers the Model Centric AI (MCAI) engine, which is deployed on the merged structured data and enabled by recurrent neural network (RNN). Representative embodiments are Time-adaptive RNN, time-aware RNN, Long Short-Term Memory (LSTM) Networks, etc. The graphical or sequential layout of the problem, as described below, make the incorporation of RNN particularly relevant here. Overall, this brick recapitulates the hidden structure discovery described under the sub-brick G.5, however in a time-aware manner. In brick O, the problem-solving goal is for both classification and regression. The models are iterated over based on the estimated classification accuracy and/or regression loss.

For the patient journey, modeling the data in each microlake is projected to a graphical structure as represented by a sequence of interactions in the A×P ecosystem (equation below). For the purposes of MCAI, the nodes are referred to as features and define the feature-space and the evolution of the model is time aware. An example goal here is to predict the trajectory of the patient's health journey. Another example is to model and predict multiple health trajectories using RNN to compare and contrast outcomes under the single physician discontinuity versus multi-physician treatment across discontinuity approaches.

The multi-trajectory patient health journey modeling can be liked to a road network which is a directed graph with vertices or cross-roads and edges or road segments, where the putative best option could be based on the trajectory with the highest probability. The treatment with and without consideration of the care disc-continuum introduced by the switching of physicians can be modeled as the predicted health trajectory of the patient based on complete and incomplete observed trajectories, respectively, very similar to that for autonomous vehicles.

Iρ→f[Aρ,Pρ,(ρ)]

For diagnosis and/or treatment modeling the linkage (equation below) as in the physician lakescape will be invoked. The linkages will connect to the appropriate patients, which now incorporate the graphical structure as defined by the patient's timeline. An example goal here is to model the equivalence of diagnoses and/or treatments across the physician landscape where there are patient overlaps.

∪ₖ₌₁^{K}Sₚ_{ₖ}⊃((A₁×P₁,A₂×P₁ . . . ,Aₙ×P₁),(A₁×P₂,A₂×P₂ . . . ,Aₘ×P₂), . . . (A₁×Pₖ,A₂×Pₖ . . . ,Aₘ×Pₖ))

The success of the models, is always dependent to a large part on the diversity of the training. An example of estimating the performance of the model would be to minimize the errors observed in the distributions between the truth and the predicted.

Brick R powers the insights generation engine which summarizes the output of the MCAI, and couples the output (insight summaries) with Natural Language Interpretation (NLI) and Natural Language generation (NLG) to provide recommendations/insights to physician-entered queries. The recommendations may include differential diagnosis, actions to be taken by the physician, prescriptions, etc. Potential diagnosis may include a listing of possible conditions for causing a patient's symptoms and associated likelihood expressed in various forms (e.g. numerical percentage, assigned grading, etc.) Recommendations may be generated in various health contexts such as, but not limited to, remote health, maternity health, minority health, etc. In alternate example implementations, bricks B-R of the technology cascade are triggered/initiated only after a physician has entered a query pertaining to a patient.

In some example implementations, a personalized physician clinical care prediction model predicts physician's treatment response based on patterns in patient cohort—including but not limited to demographics, history, symptoms, etc.—disease specialty, clinical care paradigm. The system would posit an automated prediction of their own assessment. This augmented intelligence system would provide a mutable set of recommendations vis-à-vis follow-up questions, test modalities, differential diagnoses, treatment bit Rx and non-Rx, potential lifestyle changes, etc. One way of doing this would be to identify the trajectories of physician practice/treatment with highest probabilities. The physician would maintain the ultimate editorial authority.

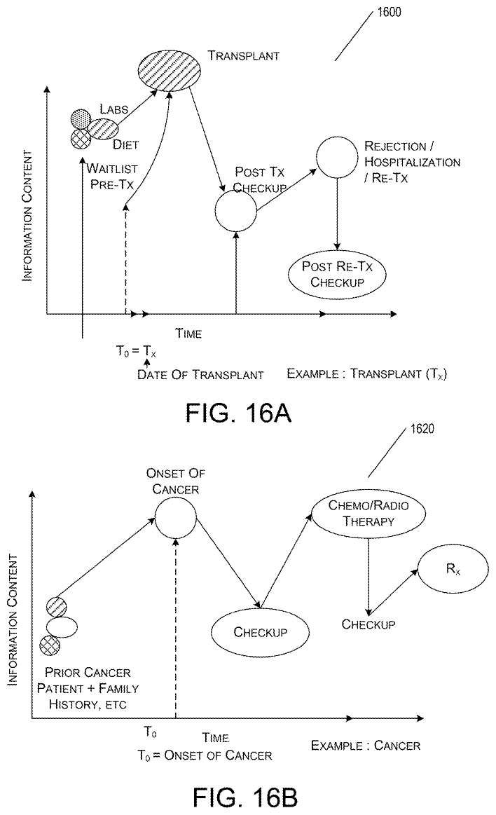

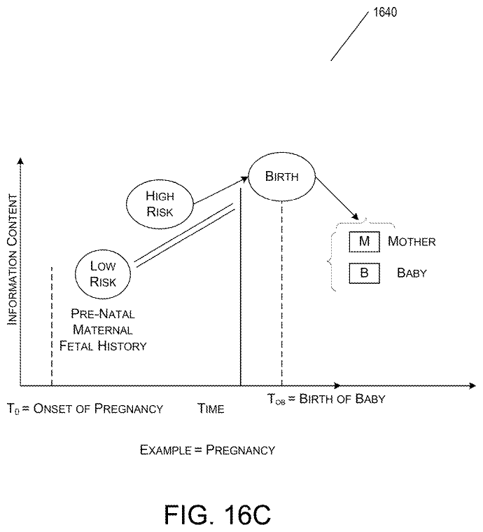

FIGS. 16(A)-(C) illustrate example insights/recommendations generated by Brick R, in accordance with an example implementation. The insights based on models which are de-identified can be world readable, and model sharing can also be GDPR compliant. The sharing of both the model and the insight facilitates a system to equalize access to care.

FIG. 16(A) illustrates insight/recommendation generation process 1600 associated with transplant related use case. Information such as patient's health history, diet, etc., serve as the initial input to the system. These inputs are then synthesized by a physician to determine transplant feasibility, which may ultimately lead to organ transplant for the patient. The physician may then order post-transplant tests (e.g. blood tests, etc.) in a post-transplant checkup to verify organ functions and that there is no rejection of the transplanted organ.

The results from the post-transplant checkup are then fed into the system where the initial inputs and the results constitute inputs to the MCAI, which generates diagnosis differential where each diagnosis is associated with a predictive probability. The probabilities are normalized across all the diagnoses in the differential with a cumulative sum of 100%. A ranked list of diagnoses together with the probabilistic risk of each diagnoses are then presented to the physician for further determinations. For example, the system may estimate risk of transplant patient going into rejection, risk of hospitalization, re-transplant needs, and/or biopsy needs as outputs of the MCAI. The physician may then determine an action to take based on the outputs (e.g. re-transplant) and perform additional follow-up actions (e.g. post re-transplant checkup).

FIG. 16(B) illustrates insight/recommendation generation process 1620 associated with cancer related use case. Information such as patient's prior cancer history, prior cancer treatment, family history, etc., serve as the initial input to the system. These inputs are then synthesized by the physician on check up to determine a treatment method, for example, chemotherapy, radiation therapy, etc. Outcome/result of the treatment can then be determined at a follow-up checkup.

The outcome/result can then be channeled into the system where the initial inputs and the outcome/result constitute inputs to the MCAI, which generates diagnosis differential where each diagnosis is associated with a predictive probability. The probabilities are normalized across all the diagnoses in the differential with a cumulative sum of 100%. A ranked list of diagnoses together with the probabilistic risk of each diagnoses are then presented to the physician for further determinations. For example, the system may estimate risk for early onset of cancer(s) as output of the MCAI.

FIG. 16(C) illustrates insight/recommendation generation process 1640 associated with pregnancy related use case. Information such as patient(s) vitals, symptoms, medical history, outcome of the physical examination performed at the given time, active and past history of medication, allergies, current diet along with dietary restrictions, exercise status, status of mental faculties, etc., serve as the initial input to the system. These inputs are then synthesized by the physician to order medical tests, for example, complete blood panel, imaging, etc., which then generate test results.

The results from these tests are then fed into the system where the initial inputs and the results constitute inputs to the MCAI, which generates diagnosis differential where each diagnosis is associated with a predictive probability. The probabilities are normalized across all the diagnoses in the differential with a cumulative sum of 100%. A ranked list of diagnoses together with the probabilistic risk of each diagnoses are then presented to the physician for further determinations. For example, the system may estimate the risk of preeclampsia as output of the MCAI.

An example outcome: A personalized physician clinical care prediction model: predict physician's treatment response based on patterns in patient cohort—including but not limited to demographics, history, symptoms, etc.—disease specialty, clinical care paradigm. The system would posit an automated prediction of their own assessment. This augmented intelligence system would provide a mutable set of recommendations vis-à-vis follow-up questions, test modalities, differential diagnoses, treatment bit Rx and non-Rx, potential lifestyle changes, etc. One way of doing this would be to identify the trajectories of physician practice/treatment with highest probabilities. The physician would maintain the ultimate editorial authority.

Actionable insights derived from these models can be shared across a physician marketplace/forum. The framework enables physicians to compare their decisions against posted models and insights. Physicians can compare their decisions, against posted models/insights. Physicians can adjust models for local prevalence rates of diseases and then compare. These actionable insights sharing approach would fuel and/or accelerate care efficiency and innovation without data management burden and address medical caregiver burnout.

The insights engine will be an effective tool in emergent care settings where the findings can be circulated in natural language across the entire team responsible for patient care coordination. The unbiased mechanism of differential diagnoses or hypotheses generation would highlight confirmation biases and be effective in ameliorating potential misses and misdiagnoses. The insight engine is also an effective tool for ER/EDs to combat bounce-backs and readmissions reduce cost of care by moving care to primary care settings.

The insight engine can facilitate a gamification model for philanthropy in the medical community. This can be established via physician leaderboards. Various embodiments of this might include ranking of physicians in terms of insights contribution to the field—and this recognition can motivate physicians to opt-in to the marketplace. It can also provide motivation to the hospitals to opt-in to the system and uphold their Center of Excellence (CoE) status. Additionally, the insight engine provides the framework for a scalable B2C marketplace model for anyone in need of first or second opinions.

The role and power of the actionable insight generation engine in equalizing access to healthcare is undeniable. The occurrence of COVID-19 in 2020 is a watershed event in highlighting inequity of access. It highlighted the drivers of this inequity are social determinants; extreme poverty rose for the 1st time in over 20 years; 8M children (<18 yo) lost a parent/primary care-giver. This marketplace will enable and maximize access to the lowest income, highest risk patient. It will enable equal access to care—irrespective of social determinants, income, location, education, technology.

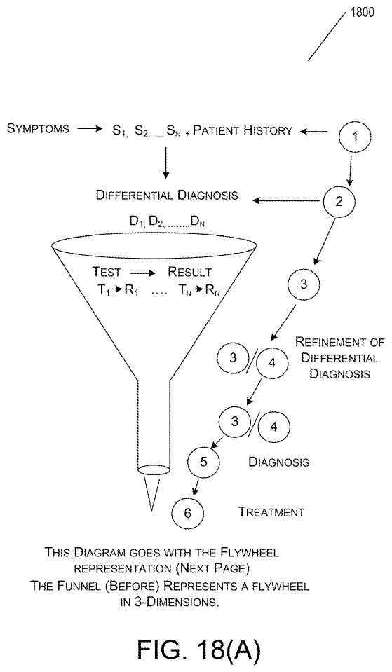

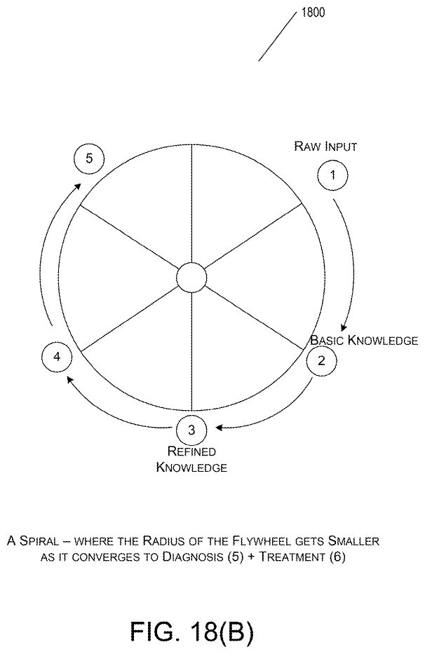

FIGS. 18(A)-(B) illustrate a flywheel representing the healthcare data conversion and insight generation system. As more knowledge about the patient and/or physician is derived through the combination of the DCAI, the MCAI, and the VIBGYOR technology cascade, the system/flywheel 1800 gains speed with each rotation/iteration (e.g. raw input, basic knowledge application, knowledge refinement, diagnosis, treatment, etc.). Momentum is gained with increasing rotation/iteration, which allows the system/flywheel 1800 to move with increased velocity. By building up on the work done previously, the system/flywheel becomes increasingly more efficient without requiring the user to expand more energy or effort, thus becoming more autonomous/self-sustaining in nature. As illustrated in FIGS. 18(A)-(B), the efficiency of the system would translate to better patient care at the top-most level, specifically, generation of better or more targeted actionable insights and/or risk prediction of a disease or outcome with higher precision and accuracy.

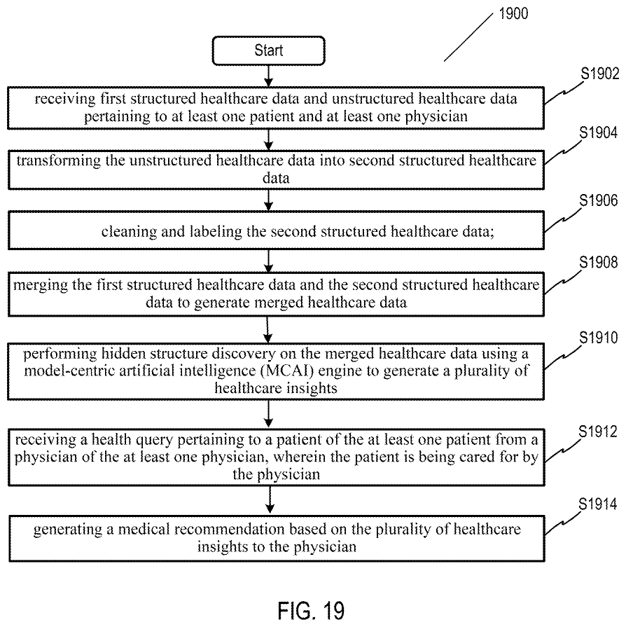

FIG. 19 illustrates an example process flow 1900 for generating healthcare insights and medical recommendation, in accordance with an example implementation. The process beings at step S1902 where first structured healthcare data and unstructured healthcare data pertaining to at least one patient and at least one physician are received. At step S1904, the unstructured healthcare data is transformed into second structured healthcare data. At step S1906, the second structured healthcare data is cleaned and labeled. At step S1908, the first structured healthcare data and the second structured healthcare data are merged to generate merged healthcare data.

The process then continues to step S1910 where hidden structure discovery is performed on the merged healthcare data using a model-centric artificial intelligence (MCAI) engine to generate a plurality of healthcare insights. At step S1912, receiving a health query pertaining to a patient of the at least one patient from a physician of the at least one physician is received (the patient being under the care of the physician). At step S1914, generating a medical recommendation generated based on the plurality of healthcare insights to the requesting physician.

The foregoing example implementation may have various benefits and advantages. For example, generation of recommendations and actionable insights and/or risk prediction of a disease or outcome with higher precision and accuracy. The system may also identify potential errors in a physician's treatment approach based on any adverse outcomes observed within or across patients under their treatment and by contrasting the treatment approaches across physicians. In addition, continuum of patient care across physician discontinuities may also be provided with ease.

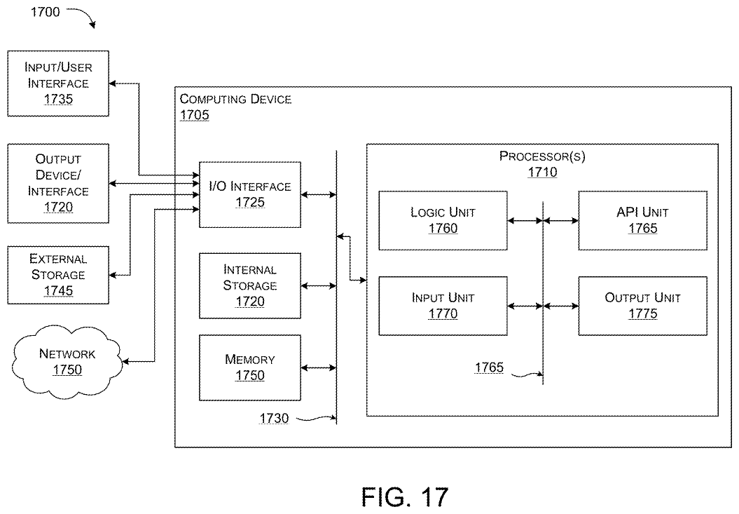

FIG. 17 illustrates an example computing environment with an example computer device suitable for use in some example implementations. Computer device 1705 in computing environment 1700 can include one or more processing units, cores, or processors 1710, memory 1715 (e.g., RAM, ROM, and/or the like), internal storage 1720 (e.g., magnetic, optical, solid-state storage, and/or organic), and/or IO interface 1725, any of which can be coupled on a communication mechanism or bus 1730 for communicating information or embedded in the computer device 1705. IO interface 1725 is also configured to receive images from cameras or provide images to projectors or displays, depending on the desired implementation.

Computer device 1705 can be communicatively coupled to input/user interface 1735 and output device/interface 1740. Either one or both of the input/user interface 1735 and output device/interface 1740 can be a wired or wireless interface and can be detachable. Input/user interface 1735 may include any device, component, sensor, or interface, physical or virtual, that can be used to provide input (e.g., buttons, touch-screen interface, keyboard, a pointing/cursor control, microphone, camera, braille, motion sensor, accelerometer, optical reader, and/or the like). Output device/interface 1740 may include a display, television, monitor, printer, speaker, braille, or the like. In some example implementations, input/user interface 1735 and output device/interface 1740 can be embedded with or physically coupled to the computer device 1705. In other example implementations, other computer devices may function as or provide the functions of input/user interface 1735 and output device/interface 1740 for a computer device 1705.

Examples of computer device 1705 may include, but are not limited to, highly mobile devices (e.g., smartphones, devices in vehicles and other machines, devices carried by humans and animals, and the like), mobile devices (e.g., tablets, notebooks, laptops, personal computers, portable televisions, radios, and the like), and devices not designed for mobility (e.g., desktop computers, other computers, information kiosks, televisions with one or more processors embedded therein and/or coupled thereto, radios, and the like).

Computer device 1705 can be communicatively coupled (e.g., via IO interface 1725) to external storage 1745 and network 1750 for communicating with any number of networked components, devices, and systems, including one or more computer devices of the same or different configuration. Computer device 1705 or any connected computer device can be functioning as, providing services of, or referred to as a server, client, thin server, general machine, special-purpose machine, or another label.

IO interface 1725 can include but is not limited to, wired and/or wireless interfaces using any communication or IO protocols or standards (e.g., Ethernet, 802.11x, Universal System Bus, WiMax, modem, a cellular network protocol, and the like) for communicating information to and/or from at least all the connected components, devices, and network in computing environment 1700. Network 1750 can be any network or combination of networks (e.g., the Internet, local area network, wide area network, a telephonic network, a cellular network, satellite network, and the like).

Computer device 1705 can use and/or communicate using computer-usable or computer readable media, including transitory media and non-transitory media. Transitory media include transmission media (e.g., metal cables, fiber optics), signals, carrier waves, and the like. Non-transitory media include magnetic media (e.g., disks and tapes), optical media (e.g., CD ROM, digital video disks, Blu-ray disks), solid-state media (e.g., RAM, ROM, flash memory, solid-state storage), and other non-volatile storage or memory.

Computer device 1705 can be used to implement techniques, methods, applications, processes, or computer-executable instructions in some example computing environments. Computer-executable instructions can be retrieved from transitory media, and stored on and retrieved from non-transitory media. The executable instructions can originate from one or more of any programming, scripting, and machine languages (e.g., C, C++, C #, Java, Visual Basic, Python, Perl, JavaScript, and others).

Processor(s) 1710 can execute under any operating system (OS) (not shown), in a native or virtual environment. One or more applications can be deployed that include logic unit 1760, application programming interface (API) unit 1765, input unit 1770, output unit 1775, and inter-unit communication mechanism 1795 for the different units to communicate with each other, with the OS, and with other applications (not shown). The described units and elements can be varied in design, function, configuration, or implementation and are not limited to the descriptions provided. Processor(s) 1710 can be in the form of hardware processors such as central processing units (CPUs) or in a combination of hardware and software units.

In some example implementations, when information or an execution instruction is received by API unit 1765, it may be communicated to one or more other units (e.g., logic unit 1760, input unit 1770, output unit 1775). In some instances, logic unit 1760 may be configured to control the information flow among the units and direct the services provided by API unit 1765, the input unit 1770, the output unit 1775, in some example implementations described above. For example, the flow of one or more processes or implementations may be controlled by logic unit 1760 alone or in conjunction with API unit 1765. The input unit 1770 may be configured to obtain input for the calculations described in the example implementations, and the output unit 1775 may be configured to provide an output based on the calculations described in example implementations.

Processor(s) 1710 can be configured to receive first structured healthcare data and unstructured healthcare data pertaining to at least one patient and at least one physician as shown in FIG. 19. The processor(s) 1710 may also be configured to transform the unstructured healthcare data into second structured healthcare data as shown in FIG. 19. The processor(s) 1710 may also be configured to clean and label the second structured healthcare data as shown in FIG. 19. The processor(s) 1710 may also be configured to merge the first structured healthcare data and the second structured healthcare data to generate merged healthcare data as shown in FIG. 19. The processor(s) 1710 may also be configured to perform hidden structure discovery on the merged healthcare data using a model-centric artificial intelligence (MCAI) engine to generate a plurality of healthcare insights as shown in FIG. 19. The processor(s) 1710 may also be configured to receive a health query pertaining to a patient of the at least one patient from a physician of the at least one physician, wherein the patient is being cared for by the physician as shown in FIG. 19. The processor(s) 1710 may also be configured to generate a medical recommendation based on the plurality of healthcare insights to the physician as shown in FIG. 19.

The processor(s) 1710 may also be configured to generate a patient data lake and a physician data lake from the first structured healthcare data and the unstructured healthcare data, wherein the patient data lake comprises at least one patient micro data lake and the physician data lake comprises at least one physician micro data lake as shown in FIGS. 13 and 14.

The processor(s) 1710 may also be configured to create linkage between the patient data lake and the physician data lake through data element tagging, wherein each patient micro data lake of the at least one patient micro data lake corresponds to a patient of the at least one patient, and each physician micro data lake of the at least one physician micro data lake corresponds to a physician of the at least one physician as shown in FIGS. 13 and 14.

The processor(s) 1710 may also be configured to generate a physician-specific thesaurus for each of the at least one physician micro data lake as shown in FIG. 9. The processor(s) 1710 may also be configured to generate a multi-physician thesaurus from the physician data lake as shown in FIG. 9.

Some portions of the detailed description are presented in terms of algorithms and symbolic representations of operations within a computer. These algorithmic descriptions and symbolic representations are the means used by those skilled in the data processing arts to convey the essence of their innovations to others skilled in the art. An algorithm is a series of defined steps leading to a desired end state or result. In example implementations, the steps carried out require physical manipulations of tangible quantities for achieving a tangible result.

Unless specifically stated otherwise, as apparent from the discussion, it is appreciated that throughout the description, discussions utilizing terms such as “processing,” “computing,” “calculating,” “determining,” “displaying,” or the like, can include the actions and processes of a computer system or other information processing device that manipulates and transforms data represented as physical (electronic) quantities within the computer system's registers and memories into other data similarly represented as physical quantities within the computer system's memories or registers or other information storage, transmission or display devices.

Example implementations may also relate to an apparatus for performing the operations herein. This apparatus may be specially constructed for the required purposes, or it may include one or more general-purpose computers selectively activated or reconfigured by one or more computer programs. Such computer programs may be stored in a computer readable medium, such as a computer readable storage medium or a computer readable signal medium. A computer readable storage medium may involve tangible mediums such as, but not limited to optical disks, magnetic disks, read-only memories, random access memories, solid-state devices, and drives, or any other types of tangible or non-transitory media suitable for storing electronic information. A computer readable signal medium may include mediums such as carrier waves. The algorithms and displays presented herein are not inherently related to any particular computer or other apparatus. Computer programs can involve pure software implementations that involve instructions that perform the operations of the desired implementation.

Various general-purpose systems may be used with programs and modules in accordance with the examples herein, or it may prove convenient to construct a more specialized apparatus to perform desired method steps. In addition, the example implementations are not described with reference to any particular programming language. It will be appreciated that a variety of programming languages may be used to implement the teachings of the example implementations as described herein. The instructions of the programming language(s) may be executed by one or more processing devices, e.g., central processing units (CPUs), processors, or controllers.

As is known in the art, the operations described above can be performed by hardware, software, or some combination of software and hardware. Various aspects of the example implementations may be implemented using circuits and logic devices (hardware), while other aspects may be implemented using instructions stored on a machine-readable medium (software), which if executed by a processor, would cause the processor to perform a method to carry out implementations of the present application. Further, some example implementations of the present application may be performed solely in hardware, whereas other example implementations may be performed solely in software. Moreover, the various functions described can be performed in a single unit, or can be spread across a number of components in any number of ways. When performed by software, the methods may be executed by a processor, such as a general-purpose computer, based on instructions stored on a computer readable medium. If desired, the instructions can be stored on the medium in a compressed and/or encrypted format.

Moreover, other implementations of the present application will be apparent to those skilled in the art from consideration of the specification and practice of the teachings of the present application. Various aspects and/or components of the described example implementations may be used singly or in any combination. It is intended that the specification and example implementations be considered as examples only, with the true scope and spirit of the present application being indicated by the following claims.

What is claimed is:

1. A method for generating medical recommendation through performance of autonomous medical operations, the method comprising:
receiving, by a processor, first structured healthcare data and unstructured healthcare data pertaining to at least one patient and at least one physician;
transforming, by the processor, the unstructured healthcare data into second structured healthcare data;
cleaning and labeling, by the processor, the second structured healthcare data;
merging, by the processor, the first structured healthcare data and the second structured healthcare data to generate merged healthcare data;
performing, by the processor, hidden structure discovery on the merged healthcare data using a model-centric artificial intelligence (MCAI) engine to generate a plurality of healthcare insights;
receiving, by the processor, a health query pertaining to a patient of the at least one patient from a physician of the at least one physician, wherein the patient is being cared for by the physician; and
generating, by the processor, a medical recommendation based on the plurality of healthcare insights to the physician.

2. The method of claim 1, wherein transforming the unstructured healthcare data into the second structured healthcare data and cleaning and labeling the second structured healthcare data are performed using a data-centric artificial intelligence (DCAI) model.

3. The method of claim 1,
wherein transforming the unstructured healthcare data into the second structured healthcare data comprises transforming the unstructured healthcare data using a neuro symbolic artificial intelligence (NSAI) model; and
wherein the unstructured healthcare data comprises at least one of physician's notes, patient history, images, or videos.

4. The method of claim 1, wherein merging the first structured healthcare data and the second structured healthcare data to generate the merged healthcare data comprises merging the first structured healthcare data and the second structured healthcare data are merged along a timeline to generate the merged healthcare data.

5. The method of claim 1, wherein generating the medical recommendation based on the plurality of healthcare insights comprises identifying and summarizing at least one healthcare insight from the plurality of healthcare insights to generate insight summaries, and applying Natural Language Interpretation (NLI) and Natural Language generation (NLG) to the insight summaries to generate the medical recommendation.

6. The method of claim 1, further comprising:
generating, by the processor, a patient data lake and a physician data lake from the first structured healthcare data and the unstructured healthcare data, wherein the patient data lake comprises at least one patient micro data lake and the physician data lake comprises at least one physician micro data lake.

7. The method of claim 6, further comprising:
creating, by the processor, linkage between the patient data lake and the physician data lake through data element tagging,
wherein each patient micro data lake of the at least one patient micro data lake corresponds to a patient of the at least one patient, and each physician micro data lake of the at least one physician micro data lake corresponds to a physician of the at least one physician.

8. The method of claim 7,
wherein data element tagging segregates the at least one patient and the at least one physician; and
wherein the at least one patient is segregated through Social Security Number (SSN) and/or Medical Record Number (MRN), and the at least one physician is segregated through National Provider Identifier (NPI).

9. The method of claim 6, further comprising:
generating a physician-specific thesaurus for each of the at least one physician micro data lake; and
generating a multi-physician thesaurus from the physician data lake.

10. The method of claim 9, wherein performing the hidden structure discovery on the merged healthcare data comprises:
comparing each physician-specific thesaurus against the multi-physician thesaurus and using Natural Language Inference (NLI) to find similarities and/or differences between hypothesis.

11. A system for generating medical recommendation through performance of autonomous medical operations, the system comprising:
a database; and
a processor in communication with the database, the processor is configured to:
receiving first structured healthcare data and unstructured healthcare data pertaining to at least one patient and at least one physician;
storing the first structured healthcare data and the unstructured healthcare data in the database;
transforming the unstructured healthcare data into second structured healthcare data;
cleaning and labeling the second structured healthcare data;
merging the first structured healthcare data and the second structured healthcare data to generate merged healthcare data;
performing hidden structure discovery on the merged healthcare data using a model-centric artificial intelligence (MCAI) engine to generate a plurality of healthcare insights;
receiving a health query pertaining to a patient of the at least one patient from a physician of the at least one physician, wherein the patient is being cared for by the physician; and
generating a medical recommendation based on the plurality of healthcare insights to the physician.

12. The system of claim 11, wherein transforming the unstructured healthcare data into the second structured healthcare data and cleaning and labeling the second structured healthcare data are performed using a data-centric artificial intelligence (DCAI) model.

13. The system of claim 11,
wherein transforming the unstructured healthcare data into the second structured healthcare data comprises transforming the unstructured healthcare data using a neuro symbolic artificial intelligence (NSAI) model; and
wherein the unstructured healthcare data comprises at least one of physician's notes, patient history, images, or videos.

14. The system of claim 11, wherein merging the first structured healthcare data and the second structured healthcare data to generate the merged healthcare data comprises merging the first structured healthcare data and the second structured healthcare data are merged along a timeline to generate the merged healthcare data.

15. The system of claim 11, wherein generating the medical recommendation based on the plurality of healthcare insights comprises identifying and summarizing at least one healthcare insight from the plurality of healthcare insights to generate insight summaries, and applying Natural Language Interpretation (NLI) and Natural Language generation (NLG) to the insight summaries to generate the medical recommendation.

16. The system of claim 11, wherein the processor is further configured to:
generate a patient data lake and a physician data lake from the first structured healthcare data and the unstructured healthcare data, wherein the patient data lake comprises at least one patient micro data lake and the physician data lake comprises at least one physician micro data lake.

17. The system of claim 16, further comprising:
creating, by the processor, linkage between the patient data lake and the physician data lake through data element tagging,
wherein each patient micro data lake of the at least one patient micro data lake corresponds to a patient of the at least one patient, and each physician micro data lake of the at least one physician micro data lake corresponds to a physician of the at least one physician.

18. The system of claim 17,
wherein data element tagging segregates the at least one patient and the at least one physician; and
wherein the at least one patient is segregated through Social Security Number (SSN) and/or Medical Record Number (MRN), and the at least one physician is segregated through National Provider Identifier (NPI).

19. The system of claim 16, wherein the processor is further configured to:
generate a physician-specific thesaurus for each of the at least one physician micro data lake; and
generate a multi-physician thesaurus from the physician data lake.

20. The system of claim 19, wherein the processor is configured to perform the hidden structure discovery on the merged healthcare data by:
comparing each physician-specific thesaurus against the multi-physician thesaurus and using Natural Language Inference (NLI) to find similarities and/or differences between hypothesis.
